(19) United States
(12) Patent Application Publication
(10) Pub. No.: US 2010/0275934 A1
(43) Pub. Date: Nov. 4, 2010

(54) Magnetic Method and System for Locating A Target

(75) Inventor: Hanan Keren, Kfar Sava (IL)

Correspondence Address: HOUSTON ELISEEVA, 420 BEDFORD ST, SUITE 155, LEXINGTON, MA, 02420, US

(73) Assignee: TOPSHOOTER MEDICAL IMRI LTD., Kfar Sava (IL)

(21) Appl. No.: 12/832,687

(22) Filed: Jul. 8, 2010

Related U.S. Application Data
(63) Continuation of application No. PCT/IL2009/000029, filed on Jan. 8, 2009.
(60) Provisional application No. 61/006,346, filed on Jan. 8, 2008.
(60) Provisional application No. 61/071,582, filed on May 7, 2008.

(51) Int. Cl. A61B 19/00; G06F 15/00; G01B 7/004; H01H 47/22

(52) U.S. Cl. 128/899; 702/152; 324/228; 361/147

(57) ABSTRACT

The present invention provides an accurate and real-time acquisition and monitoring of three-dimensional location information about a target, in particular a target inside a subject's body during a medical procedure. This is achieved in accordance with the invention by marking the target location by a small-size location marker, which can be detected and located with high signal-to-noise ratio of the detection by a detection system located at a distance from the target. Provided by the invention is a location marker, a target location system utilizing such marker, and also a novel antenna system suitable to be used in the target location system. The marker of the present invention is a passive electronic device, which “responds” to an external high radio frequency electromagnetic field by a periodic time pattern of a single (certain fixed value) relatively low frequency, as compared to the known devices of the kind specified emitting a frequency coded signal.

### FIELD OF THE INVENTION

The present invention is in the field of magnetic techniques for target location and relates to various aspects involved in such techniques, including a marker, an antenna array and detection system.

### BACKGROUND OF THE INVENTION

Techniques for identifying a target and determining the target location are used in various applications including inter alia security and medical applications. Conventional techniques of the kind specified typically utilize appropriate markers responding to an interrogating field.

Medical procedures often require locating and treating areas within a subject's (patient's) body. Imaging systems, including X-ray, MRI, CT, and ultrasound have been used to assist in locating areas or particular targets within the body. While the imaging systems can be very useful in some situations, they might be unusable or difficult to use in certain procedures to provide real time three dimensional location information about a target.

Many noninvasive medical procedures, such as radiation therapy and surgical procedures require precise location information about the target to minimize the extent of collateral damage to healthy tissue around the target. Markers have been used to locate targets on and in a patient's body in preparation for a medical procedure.

One kind of “marking” the target location consists of using gold fiducials, which are solid, inert, metal beads that can be implanted in a patient at or near a tumor or other target that may be difficult to accurately detect using conventional imaging systems. Markers of another kind utilize a radioactive source (source of ion radiation); yet other known markers are electro magnetic markers.

Various identifiable markers and marker detection systems are disclosed for example in WO 98/30166, WO 02/16965; US 2002/193685; WO06043276; US 2006/0187059; and EP 0696744.

### GENERAL DESCRIPTION

There is a need in the art to facilitate the accurate and real-time acquisition and monitoring of three-dimensional location information about a target, in particular a target inside a subject's body during a medical procedure. The target may be a defined portion of tissue or organ within the body or a portion of a medical tool or device invading the body (e.g. a catheter, endoscope or needle). This is achieved in accordance with the invention by marking the target location by a small-size location marker, as will be described below, which can be detected and located with high signal-to-noise ratio of the detection by a detection system located at a distance from the target. Provided by the invention is a location marker, a target location system utilizing such marker, and also a novel antenna system suitable to be used in the target location system. While the aspects of the invention are described in the present application with particular reference to their medical use, it should be understood that the invention is not limited to this specific application.

Considering the medical applications, the marker of the present invention can be implanted in a patient's body at or near a selected target, such as a tumor, for guiding radiation therapy procedure. The marker can be used as well for guidance of probes such as catheters, endoscopes, needles or for location colonoscopy. More specifically, parts of intrabody objects such as orthopedic implants, various invasive medical instruments, catheters, endoscopes, gastroscopes, bronchoscopes, biopsy tools and needles can be manufactured using the disclosed markers. For example, the marker of the present invention may be used in a catheter-based procedure for diagnosis or treatment of conditions of the heart, such as heart failure. The probes that may be guided by the marker of the present invention include balloon angioplasty catheters, catheters with laser-, electrical- or cryo-ablation characteristics, catheters having ultrasound imaging heads, probes used for nearly incisionless-surgery or diagnosis, endoscopes and catheter used for electrophysiological mapping (EPM) and ablation.

The marker can be used, as well, in the diagnosis or treatment of intravascular ailments, which may involve angioplasty or atherectomy. The principles of the marker may also be applied, mutatis mutandis, in position-sensing systems for the diagnosis or treatment of other body structures, such as the brain, spine, skeletal joints, urinary bladder, gastrointestinal tract, prostrate, and uterus.

It should be understood that conventionally such invasive medical instrument may be guided by a locating system connectable via wires. By using the marker of the present invention, associated with any invasive medical instrument, the medical instrument may be guided wireless.

The marker of the present invention is a passive electronic device. The term “passive device” means that the device has no internal power source (i.e. “battery free” device), being activated by an external radio-frequency electromagnetic field, and the device is essentially not emitting radiation as compared to that of said external field.

The marker of the present invention “responds” to an external high radio frequency electromagnetic field by a periodic time pattern of a single (certain fixed value) relatively low frequency, as compared to the known devices of the kind specified emitting a frequency coded signal.

Provided by the invention is, thus, a location marker comprising an electronic circuit energizable by a high radio frequency electromagnetic field i.e. being switchable from its normally inoperative state into its operative state by a high radio frequency electromagnetic field. Upon being so energized, the electronic circuit is switched into an operative state in which the marker generates a predefined periodic distortion pattern of a certain fixed frequency relatively low as compared to the high radio frequency thus inducing a corresponding modulation of said electromagnetic field. In other words, the marker of the present invention is configured to be energized (powered) by an external alternating (AC) magnetic field, to generate a local pulsed magnetic field within the external magnetic field region of a pulse frequency having a low-frequency time modulation (e.g. 10 Hz), thus causing detectable load modulation on the external magnetic field.

According to an embodiment of the invention, the high frequency/low frequency ratio is at least 2, but may reach significantly higher values up to 1000 and higher. The high frequency is typically within the range of about 50 to about 1,000 KHz, e.g. between about 100 to 150 KHz.

According to an embodiment of the invention, said low frequency is in the range of a few Hz to several tens of KHz, e.g., about 1 to 20 KHz.

According to certain embodiments of the invention, the electronic circuit comprises a magnetic component and an electronic switch unit electrically connected to said magnetic component. The magnetic component is energizable by the high frequency field to actuate the electronic switch unit which is programmed to operate in a switching mode of said predefined periodic low frequency modulation. The switching operation causes said low frequency distortion pattern of the electric current flow through the magnetic component.

In some embodiments the magnetic component comprises a ferromagnetic element (preferably with a coil) with passive and active magnetic properties. The electronic switch unit affects the active magnetic properties with said periodic low frequency distortion pattern. In some specific embodiment, the electronic circuit comprises a loading RF resonance circuit with passive and active magnetic properties and an electronic switch unit affecting the active magnetic properties in said periodic low frequency distortion pattern.

In some applications the location marker is housed in a capsule made of a biocompatible material.

In some applications the system is configured to have a plurality of location markers. In such a case the frequency and/or amplitude of the predefined periodic low frequency distortion pattern induced by each marker is different from the others, e.g. being different in the gated frequency and/or time dependence modulation amplitude.

Also provided by the invention is a system for locating a target comprising one or more location markers as described above, generating a predefined periodic pattern of a different low frequency and comprising a transceiver antenna system. The transceiver antenna system is configured for (i) generating and receiving said high frequency electromagnetic field, (ii) processing the received signal and extracting the low frequency periodic modulation component therefrom (e.g. by applying an autocorrelation function to the received signal), one for each marker, and (iii) identifying location of the marker based on the extracted component.

The transceiver antenna system typically comprises a plurality of sensing coils arranged in three arrays defining three sensing apertures located in three different non-parallel planes. To ensure minimal cross-talk between the coils of different arrays, the different arrays are located in three non-parallel planes and/or are appropriately spaced from one another.

Preferably, the interaction between adjacent sensing coils within each array of the transceiver antenna system is minimized by associating each coil with its own amplifier. Also, preferably, the sensing aperture of the sensing coil is partially overlapping with that of an adjacent coil, setting their mutual inductance, and thus the coupling, to zero.

The target location system may also comprise a magnetic field source configured and operable for generating an AC magnetic field of a frequency of about 100 KHz-150 KHz.

In some embodiments, the high radio frequency electromagnetic field has a constant frequency and peak amplitude.

The target location system of the present invention may be used for directing irradiation to a target tissue.

In some embodiments, the marker is fitted on a medical tool. The medical tool may be a catheter, an endoscope or a needle.

It is also provided a medical instrument carrying a location marker as defined above.

The marker is thus detectable by the distortion pattern of the external magnetic field being detected by the detection system. It should be noted that the marker of the present invention allows its detection with high signal to noise ratio relative to predicate, resulting in higher measured accuracy for the marker spatial position and in a relatively high flexibility regarding the possible distance between the detection system and the marker location (e.g. the treated body).

The high overall signal to noise ratio of the entire system (marker and target location system) relative to predicate can be 60 dB or even higher. This is due to the capability of the target location system for continuous monitoring of the complex distortion pattern change because it has a low gated discrete frequency as compared to the energizing frequency. In other words, the location marker produces a distortion pattern having a single low frequency, rather than a frequency coded signal.

It should be noted that the continuous monitoring of the distortion pattern enables a real-time determination of 3D location of a marker having an active switching frequency down to few Hertz. Conventionally, the active switching frequency of such markers is about 300-500 KHz because of the relaxation (the time decay of the magnetization) of the sample measured after the switch-off or the switch-on of the magnetic field.

According to another broad aspect of the present invention, there is provided a transceiver antenna system comprising a magnetic field source of an alternating electromagnetic field; a plurality of sensing coils arranged in three arrays defining three sensing apertures located in three different non-parallel planes, wherein sensing apertures of locally adjacent coils in each array are partially overlapping setting their mutual inductance to zero; and, a receiver for receiving an incoming electromagnetic signal and extracting therefrom a predefined periodic signal of a low frequency, thereby enabling identification and determination of a three-dimensional location of the marker with a high signal-to-noise ratio.

Also provided by the invention is an antenna system comprising: a receiving antenna arrangement comprising three or more, typically three, phase arrays, each being typically two-dimensional, of closed-loop coils defining each a receiving aperture of substantially the same polygonal geometry, for example square geometry; said arrays being each located in a different plane, all of the planes being in a spaced-apart parallel relationship, said coils being arranged with reduced cross-talk between at least some of said coils.

In accordance with some embodiments of the invention the coils are arranged such that each coil of one array is superimposed along at least two sides thereof by coils from at least one other array with certain polygonal overlapping regions substantially small as compared to said receiving aperture.

In some embodiments the configuration is such that each coil of a first of the arrays is along two sides thereof superimposed by two coils, one from each of a second and a third of the arrays. These may be opposite sides of the coil; and/or adjacent sides of the coils.

Preferably, the coils are arranged such that the first array comprises at least one pair of coils arranged with a space between them, with the two coils of the second and third arrays being aligned with said space.

Additionally, in some embodiments of the invention, coils of each array are arranged in first and second plurality of rows along first and second perpendicular axes, respectively, such that a number of coils in the row is different by 1 from a number of coils in the locally adjacent parallel row. In some other embodiments, coils of each array are arranged in first and second plurality of rows along first and second perpendicular axes, respectively, such that each of the uppermost and lowermost rows of the first plurality of rows has n coils, and each of the intermediate rows of the first plurality has (n+1) coils.

In some embodiments of the invention the antenna system comprises a receiver unit, configured and operable for processing received signals from each of said coils and determining three-dimensional coordinates of an external source of an electromagnetic field. Said receiver unit may be configured and operable for processing received signals from each of said coils and determining three-dimensional coordinates of an external source of an electromagnetic field by a set or array of measurements taken from the coils (i.e., set of actual measurements). The detection system compares the set of actual measurements to sets of reference measurements sampled for various known locations within a bounding volume (also referred to as a localization volume). The bounding volume delimits the three-dimensional area in which the marker can be localized. A reference measurement for a known location indicates the measurements to be expected from the sensors when the marker is located at that known location.

Based on the comparisons, the detection system identifies the set of reference measurements that most closely matches the set of actual measurements. The known location of the identified set of reference measurements represents the known location that is closest to the marker location, which is referred to as the “closest known location.” The detection system then uses sets of reference measurements for known locations near the closest known location to more accurately determine the marker location when it is not actually at one of the known locations.

For simplicity and clarity, it is assumed that a single marker is being located or sensed. In some applications, multiple markers are to be detected by the same detection system, for example the markers being associated with a subject or patient. It should be noted that for obtaining accurate 3D information on the location of the treated anatomy two or preferably three implanted markers are required. In such a case, the teachings herein can easily be extended to multiple markers. Each powered marker is gated in turn at different low gated frequency and the multiple markers are detected/located simultaneously. Thus, the use of multiple markers is contemplated by the present invention. Moreover, the low gated frequency enables to detect a large number of powered markers simultaneously.

The detection system determines the marker location based on an interpolation of a set of calculated measurements from the sets of reference measurements of known locations near the closest known location. Thus, the detection system uses the set of reference measurements to find a known location that is close to the marker location to an accuracy that is dependent on the spacing of the known locations. The detection system then uses an interpolation of sets of reference measurements at known locations near the closest known location to more accurately identify the marker location at a location between the known locations.

More specifically, the receiver unit is configured and operable for processing received signals from each of said coils and determining three-dimensional coordinates of an external source of an electromagnetic field by searching for an absolute minimum to a target function value performed along traces intercepting the perpendicular axis to the phase array coils. The receiver unit is configured and operable for carrying out the following:

(i) processing received signals from each of said coils and determining three-dimensional coordinates of an external source of an electromagnetic field by determining a predicted vector data indicative of the intensities of said external source signal detected by the phase array coils in the antenna magnetic field at any field point and at any inclination of the external source relatively to each local magnetic field of each coil;

(ii) detecting the signal intensities emitted by said external source by said phase coil arrays and generating a measured data vector; and

(iii) identifying the location of said external magnetic source by defining a target function indicative of the difference between said predicted vector data and said measured data vector, the target function having an absolute minimum at the external source location and at a certain angle to the magnetic field vector produced by the phase array coils.

In some embodiments, the minimum of the target function is obtained by dividing the space around said external source to a cube of sub-volumes and by sub-dividing again in a similar manner the previous sub-volume of lowest target function, until a convergence to a low target function value is achieved.

The same arrangement of coils may, by some embodiments, be used for transmitting an exciting electromagnetic field defining a transmitting antenna arrangement comprising phase arrays of coils each generating an electromagnetic field. In this case the receiver unit preferably comprises an isolated splitter electrical circuit for filtering out an electromagnetic component of the transmitted field from the received signals that are to be processed.

The receiver unit may be configured and operable for processing received signals from each of said coils, enabling at least one of the followings: the elimination of a pre-calibration stage, a phase-shifting adjustment and a phase cancellation.

The antenna arrangement of the invention may be used in a medical system for locating a target associated with a certain location within or on a subject's body, where the target is marked by a source of electromagnetic field. In a preferred embodiment of the invention the antenna system is configured for integration in a subject support.

### BRIEF DESCRIPTION OF THE FIGURES

In order to understand the invention and to see how it may be carried out in practice, a preferred embodiment will now be described, by way of non-limiting example only, with reference to the accompanying drawings, in which:

FIG. 1A schematically illustrates the operational principles of a location marker of the present invention;

FIG. 1B shows a block diagram of the electronic circuit in the location marker, according to embodiments of the invention;

FIG. 2A schematically illustrates a target location system according to some embodiments of the invention;

FIG. 2B schematically illustrates a location marker and a target location system according to some embodiments of the invention;

FIG. 3 illustrates a schematic electrical circuit of the marker;

FIGS. 4A to 4C illustrate schematically electrical diagrams of the detection system of the present invention according to three examples, respectively;

FIGS. 5A-5D exemplify signals generated and received by the detection system: FIG. 5A shows a powering signal generated by the detection system, FIG. 5B shows a distortion pattern produced by the location marker of the present invention, FIG. 5C shows a return power signal at the detection system, and FIG. 5D shows the final detected signal;

FIGS. 6, 7 illustrate schematically electrical diagrams of the detection system of the present invention according to two more examples, respectively;

FIG. 8A illustrates more specifically an isolated RF splitter used the detection system of some embodiments;

FIGS. 8B-8D illustrate equivalent circuits associated with the power splitting effect and the isolation effect induced by the isolation splitter of FIG. 11A;

FIGS. 9-10 illustrate schematically electrical diagrams of the detection system of the present invention according to two more examples, respectively;

FIGS. 11A-11F exemplify signals generated and received by the detection system: FIG. 11A shows a powering signal generated by the detection system, FIG. 11B shows a distortion pattern produced by a location marker, FIG. 11C shows a return power signal at the detection system, FIG. 11D shows a baseband detected signal; FIG. 11E shows a return power signal after phase cancellation and FIG. 11F shows a baseband detected signal after phase cancellation;

FIG. 12 illustrates schematically yet another example of an electrical diagram of the detection system of the present invention enabling detection in a higher dynamic range;

FIGS. 13A-13D exemplify signals generated and received by the detection system: FIG. 13A shows a powering signal generated by the detection system, FIG. 13B shows a distortion pattern produced by a location marker, FIG. 13C shows a return power signal at the detection system, FIG. 13D shows the final detected signal;

FIG. 14 exemplifies the coding of a plurality of programmed markers (7 in the present example) as a function of the number of bit at RF frequency of 131,072 (=2¹⁷) Hz;

FIG. 15 illustrates schematically another example of an electrical diagram of the detection system of the present invention enabling accurate determination of the markers location;

FIGS. 16A-16C exemplify signals generated and received by the detection system of FIG. 15;

FIG. 17 illustrates an example of the configuration of the detection system of the present invention comprising an array of sensing coils;

FIG. 18 illustrates an example of the configuration of sensing coils' arrays in three orthogonal planes suitable to be used in the detection system of the present invention.

FIGS. 19A-19D illustrate layout designs of phase array coils consisting, respectively, of 10 square loops, 22 square loops, 23 square loops, 31 square loops;

FIG. 19E illustrates another layout design of phase array coils consisting of 7 octagon loops;

FIGS. 19F-19I illustrate the implementation of cross-talk reduction in the designs of FIGS. 19A-19D, respectively;

FIGS. 20A-20B illustrate the coordinates system employed in calculating the magnetic flux density detectable by phase array coils comprising rectangular coils;

FIG. 20C illustrates the compliance of a measured marker signal 20M by an antenna coil as a function of the axial displacement along the main Z-axis, with a theoretical curve 20T, for antennas with square loops of 40 cm in size;

FIG. 21A represents a predicted vector data indicative of the coil's location indexes associated with the predicted signal intensities out of the phase array of rectangular coils, in the presence of a marker in the antenna magnetic field at any field point (x,y,z) and any marker inclination (θ,φ) to the local magnetic field of each coil;

FIG. 21B illustrates the predicted signal intensities from phase array coils for the nine coils example;

FIG. 21C illustrates the search trajectories of a target function value performed along 8 traces intercepting the origin of the phase array coils;

FIGS. 21D-21F illustrate the log target function of a signal along 8 traces according to three examples, respectively; and;

FIGS. 22A and 22B illustrate two examples of the transmitting and receiving functional parts of the antenna system of the present invention.

### DETAILED DESCRIPTION OF EXEMPLARY EMBODIMENTS

While the invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

The present invention provides a novel location marker, a novel target location system and a novel antenna system. The concept of the present invention can be used in various article's identification and location applications, and is particularly useful in medical application for locating a target (e.g. a region inside a patient's body, or the distal end of a catheter or endoscope) by “marking” this location by means of associating the target location with the detectable location marker.

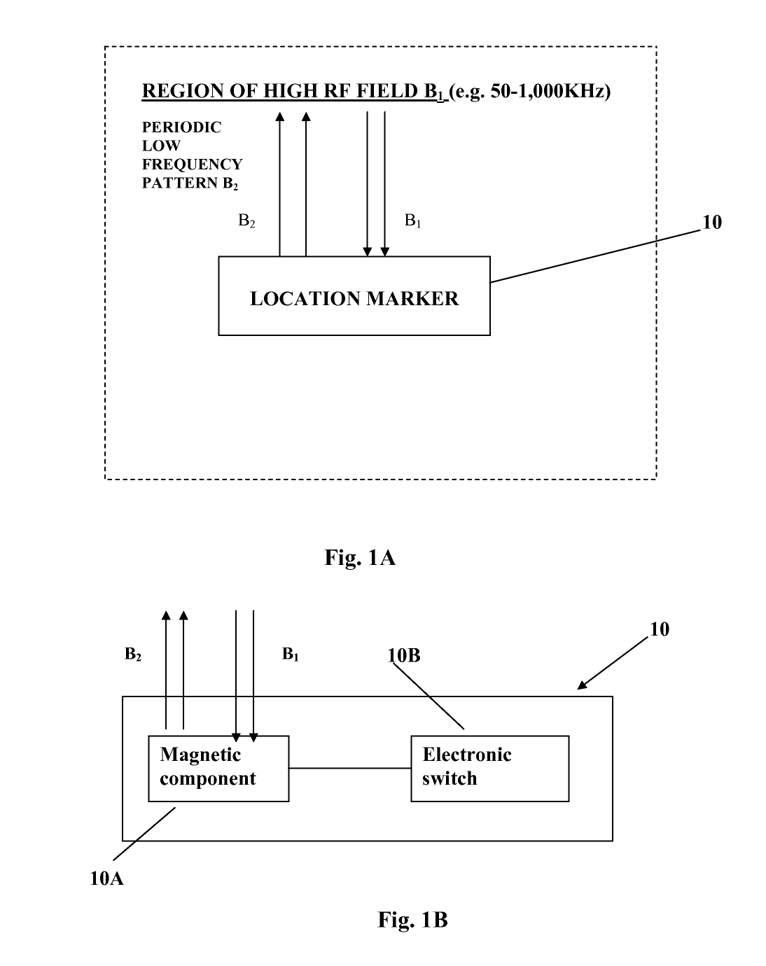

Referring to FIG. 1A, the main operational principles of a location marker 10 are schematically illustrated. The location marker 10 comprises an electronic circuit (not shown here) configured to be energizable by a high radio frequency electromagnetic field to switch into an operative state. Thus, when a region of such high radio frequency electromagnetic field B₁ is created in the vicinity of the marker or when the marker enters the region of high radio-frequency (RF) field B₁, the marker (its electronic circuit) is switched from its normal inoperative or passive state into an operative state. The electronic circuit of the marker 10 is configured such that when energized, it operates to induce a predefined periodic distortion pattern B₂ having a low-value single frequency of said electromagnetic field. The marker 10 is configured as described above, being responsive to a high radio frequency electromagnetic field and being switchable by said field into an operative state to induce a predefined periodic low frequency distortion pattern of said electromagnetic field. The marker 10 is configured to be shifted by an external alternating electromagnetic field from its normal inoperative state into its operative state to generate a local pulsed magnetic field varying with a certain low-frequency pattern of time modulation of the magnetic field value having a predefined (predetermined) periodicity. By this, the marker induces a predefined periodic low frequency distortion pattern B₂ of the external electromagnetic field.

For a given high radio frequency electromagnetic field, the marker configuration may provide the high frequency/low frequency ratio of at least 2. The high frequency may be between about 50 KHz to about 1,000 KHz, e.g. about 100-150 KHz. The low frequency may be in the range of about 1 Hz to 20 KHz.

Due to the marker operation with a single lower frequency as compared to the energizing RF frequency, and due to the predefined periodicity of the low-frequency distortion pattern induced by the marker, the marker can be detected and precisely located by the high-frequency RF detection system in real time with a high signal-to-noise ratio. This will be described more specifically further below. Moreover, the above features of the marker allow the detection system to be at least a few meters distant from the marker location without affecting the obtained accuracy of the marker detection/location.

As exemplified in FIG. 1B, the electronic circuit in the location marker 10 includes a magnetic component 10A and an electronic switch unit 10B electrically connected to the magnetic component. The magnetic component 10A is energizable by the high frequency field B₁ to actuate the electronic switch unit 10B. The latter is pre-programmed to operate in a switching mode of the predefined, periodic, low-frequency modulation (low-frequency ON/OFF mode). This switching operation causes the low-frequency distortion pattern B₂′ of the electric current flow through the magnetic component 10A, generating a magnetic field having a corresponding distortion pattern B₂ induced by the predefined periodic low frequency distortion pattern B₂′ of the electric current.

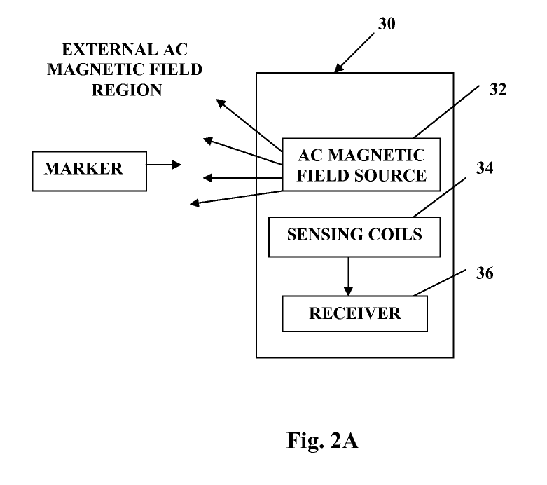

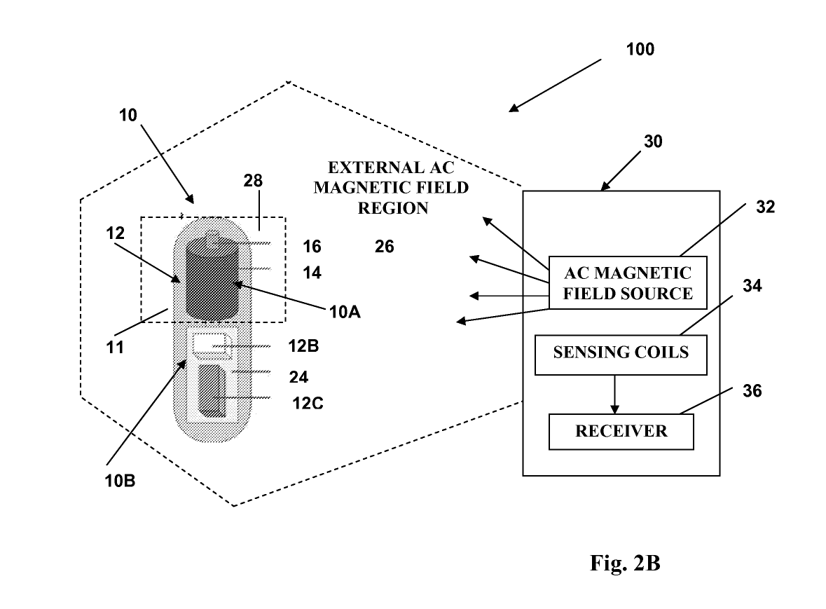

Reference is made to FIG. 2A, illustrating an example of a target location system 30 of the present invention. The system 30 is configured for at least receiving an electromagnetic field indicative of a marker response to an external “exciting” electromagnetic field. The marker response may be in the form of a distortion pattern induced by the marker in the external electromagnetic field.

In the present not limiting example, the system is configured as a transceiver antenna system for both generating the “exciting” electromagnetic field and receiving an electromagnetic field (e.g. high frequency electromagnetic field). The system 30 operates to process the received signal and extract therefrom a frequency component (e.g. low frequency periodic component) associated with the marker (at least one marker) response to the exciting electromagnetic field, and for identifying location of the marker based on the extracted component. The system 30 can be connectable (through wired or wireless communication) to a main control station (not shown here).

The system 30 includes such functional parts as an AC electromagnetic field source 32, a magnetic field sensor (sensing coils) 34, and a receiver unit 36. The functions of the high RF electromagnetic field source and the magnetic field sensor in powering (“exciting”) and detection are implemented through an antenna system configured and operable according to the invention.

Generally, any kind of antenna array can be used. Preferably, the antenna system is configured to enable the received signal translation along three non-parallels (e.g. orthogonal) axes, thus enabling determination of a point location of the local magnetic field source, i.e. of the marker. This will be described more specifically further below.

Reference is made to FIG. 2B, illustrating an example of the entire system 100 of the present invention. To facilitate understanding, the same reference numbers are used for identifying components that are common in the example of the invention. The system 100 is configured and operable for locating a target (i.e. positioning and tracking), where one or more location markers are placed at the target (e.g. in the vicinity of the target, or at a known location from the target). The target may be constituted by a region of interest (e.g. in a subject's body) or by a medical instrument. For example, one or more markers may be implanted around a region of interest. In the case of “marking” a medical instrument location, the medical instrument e.g. a catheter, carrier one or more markers thereon.

Thus, the system 100 includes one or more location markers—a single location marker 10 being shown in the present example, and a detection system 30. The marker 10 includes an electronic circuit 12 which may be located (printed or assembled) on a substrate and/or enclosed in housing (capsule), generally at 11. It should be noted that the housing may be a medically approved material (e.g. bio-ceramic), enabling the permanently implantation of the marker in a patient's body and/or placing the marker on a medical instrument for intervention the body. The electronic circuit 12 includes a magnetic component 10A or energizable unit formed by a coil 14 (e.g. copper) on a magnetic core 16 (ferrite); an electronic switch 10B electrically connected to the magnetic component 10A. In the present example, the electronic circuit 12 of the marker is configured somewhat similar to a loading RF resonance circuit suitable for use in RFID tags, namely includes a chip (electronic block) 12C electrically connected to the magnetic component 10A via a capacitor 12B. The capacitor 12B and the electronic block 12C form together the electronic switch unit 10B of the marker and can be carried by or printed on a substrate 24. According to the invention, the chip 12C is preprogrammed to, when actuated, operate in an ON/OFF switching mode with a predefined periodic low frequency, e.g. 10 Hz. It should be understood that the electronic switch unit 10B of the marker may have any other suitable configuration, for example including one or more transistors.

When the coil-on-core component 10A enters an AC electromagnetic field region 26 (high RF frequency electromagnetic field region) or when such field region is created in the vicinity of the component 10A, an electric current flow is induced in the circuit 12. This electric current, via the capacitor 12B energizes the chip 12C, which starts its ON/OFF switching mode with the low switching frequency (e.g. 10 Hz). On the other hand, when electric current flows through the component 10A, a local magnetic field is created within a magnetic field region 28 in the vicinity of the component 10A, being thus within or overlapping with the external electromagnetic field region 26.

The ON/OFF switching mode of the chip 12C affects the electric current flow through the circuit 12 and induces a corresponding low-frequency pattern of time modulation B₂ of the local magnetic field generated by the marker within the external electromagnetic field region 26. This marker-related local magnetic field provides a respective distortion pattern of the external electromagnetic field, which can be detected. The chip 12C may be configured as a simple analog switching circuit, such as the known “Schmitt Trigger” circuit set to switch for ON/OFF at the predetermined marker gating frequency. In some embodiments, the chip 12C may include a memory utility for storing data indicative of a switching mode (switching frequency).

The detection system 30 is configured for detecting and locating a “source” of the distortion pattern in the external electromagnetic field (i.e. detecting and locating the marker configured as described above), and is preferably configured also for generating this external high RF electromagnetic field.

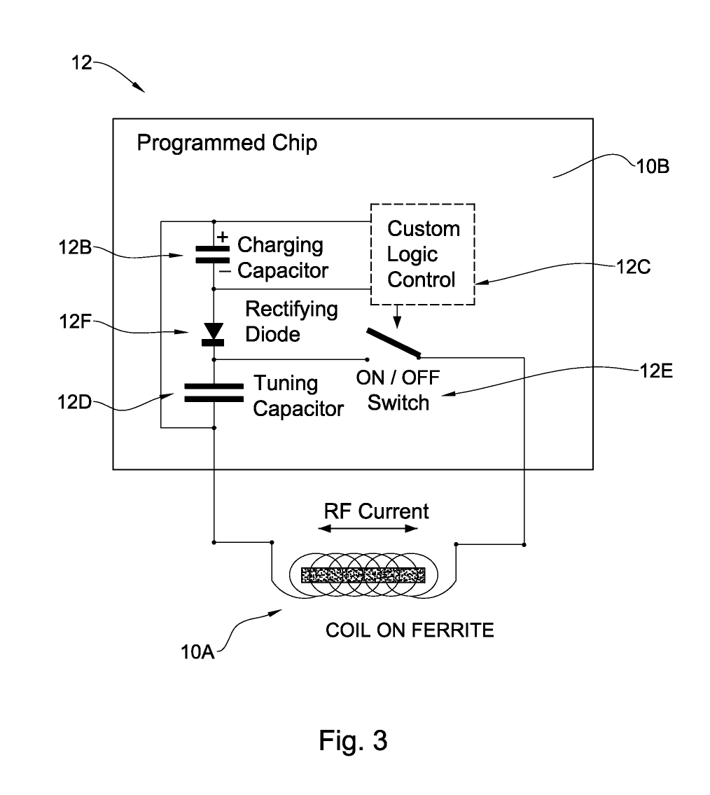

Reference is made to FIG. 3 showing a specific but not limiting example of an electronic circuit 12 of the marker. The electronic circuit 12 is formed by a magnetic component 10A (an energizable unit), formed by a coil 14 on a ferromagnetic core 16 (ferrite); a charging capacitor 12B operable to power the electronic circuit 12 upon energizing the magnetic component by the external high RF field; an optional tuning capacitor 12D; and an electronic block (custom logic control) 12C electrically connected to the magnetic component 10A via the charging capacitor 12B. The chip 12C is connected to a switch 12E and is preprogrammed to, when actuated, operate the switch 12E in an ON/OFF switching mode with a low operating frequency, e.g. 10 Hz. When the magnetic component 10A enters the high RF electromagnetic field region 26 or when such electromagnetic field region is created in the vicinity of the unit 10A, an electric current flow is induced in the circuit 12. This electric current, via the capacitors 12B, 12D and a rectifying function of a diode 12F, energizes the chip 12C, which effects the ON/OFF switching mode of the switch 12E with the low switching frequency (e.g. 10 Hz), i.e. the switch connects and disconnects with the low gated frequency.

It should be understood that the magnetic component generally includes a ferromagnetic element, i.e. ferrite core, which has dimensions (length and diameter) and permeability selected to be capable of being appropriately energizable by certain high RF electromagnetic field. In other words, the parameters of the ferromagnetic element are selected to enable the high RF electromagnetic field to induce a required electric current in the ferromagnetic element for activating the electronic switch unit. The magnetic component energizable by an RF field of about 100-150 KHz can be of appropriately small dimensions to be used in an implantable marker or to be placed on a medical tool such as needle or catheter. The use of a coil around a ferrite core makes the marker signal (low frequency distortion pattern) directional, where the ferrite's axis is in line with the reader's antenna axis.

Turning back to FIG. 2A, the above calculations of the marker location are carried by receiver unit 36, which is an electronic system configured and preprogrammed for processing data indicative of the magnetic field measured by the coils 34. Such electronic system includes an electrical circuit configured for extracting, from the measured data at the coils, a frequency component associated with the marker response, and a software utility or processing unit 50 configured for applying the above algorithm to the frequency component to determine the marker coordinates.

The following are some specific but not limiting examples of the configuration and operation of the electrical circuit for extracting the frequency component of the marker. In these examples, the detection system is configured as a transceiver antenna system, and includes a high RF electromagnetic field source 32, a sensor arrangement 34 for sensing an RF electromagnetic field; and a receiver unit 36 (hardware and software utility). In these examples, plurality of coils is used for both the field generation and sensing. The detection system generates the electromagnetic field waveform, typically of a constant energizing frequency (e.g. 125 KHz) and typically of certain constant peak amplitude.

It should be understood that some of the figures discussed below show a single coil 34 and its associated electrical circuit, but a processor unit 50 shown in the figures is associated with a plurality of such coils and their circuits for receiving and analyzing signals therefrom for determining the 3D location of the marker. As will be described more specifically further below, the sensing coils 34 are preferably arranged in three arrays defining respectively three intersecting (non-parallel) sensing apertures (e. g. located in three orthogonal planes).

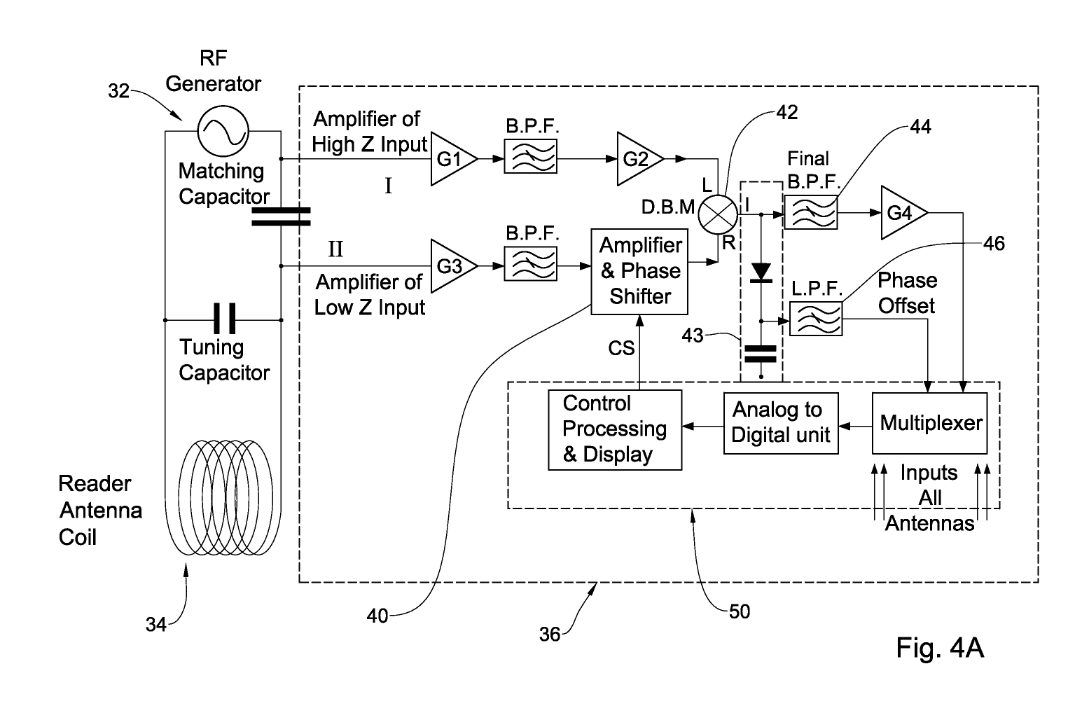

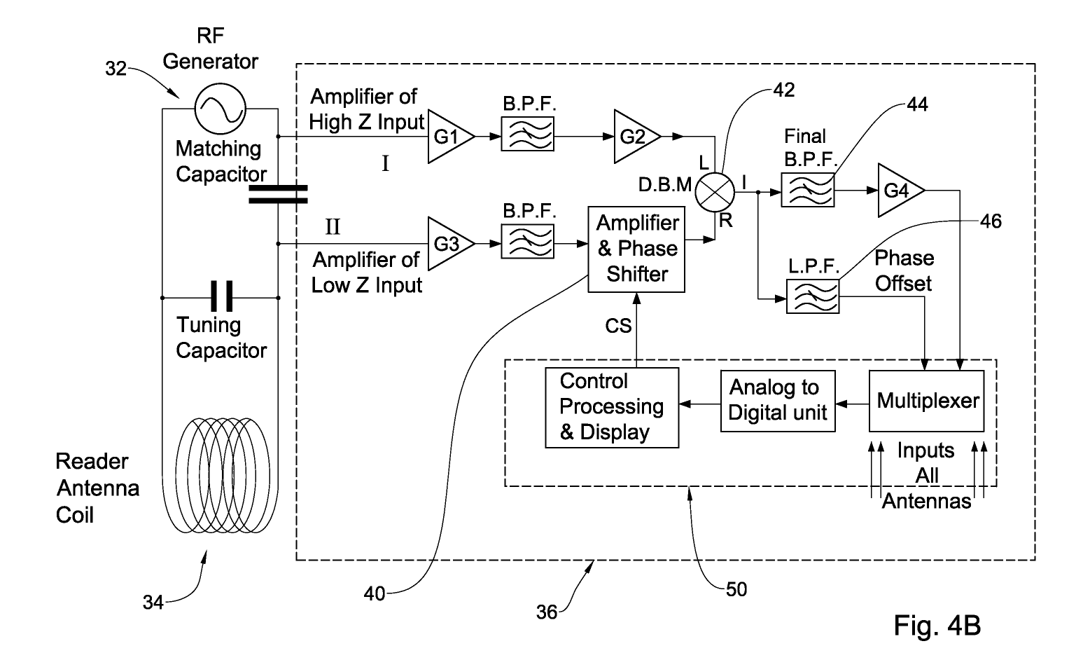

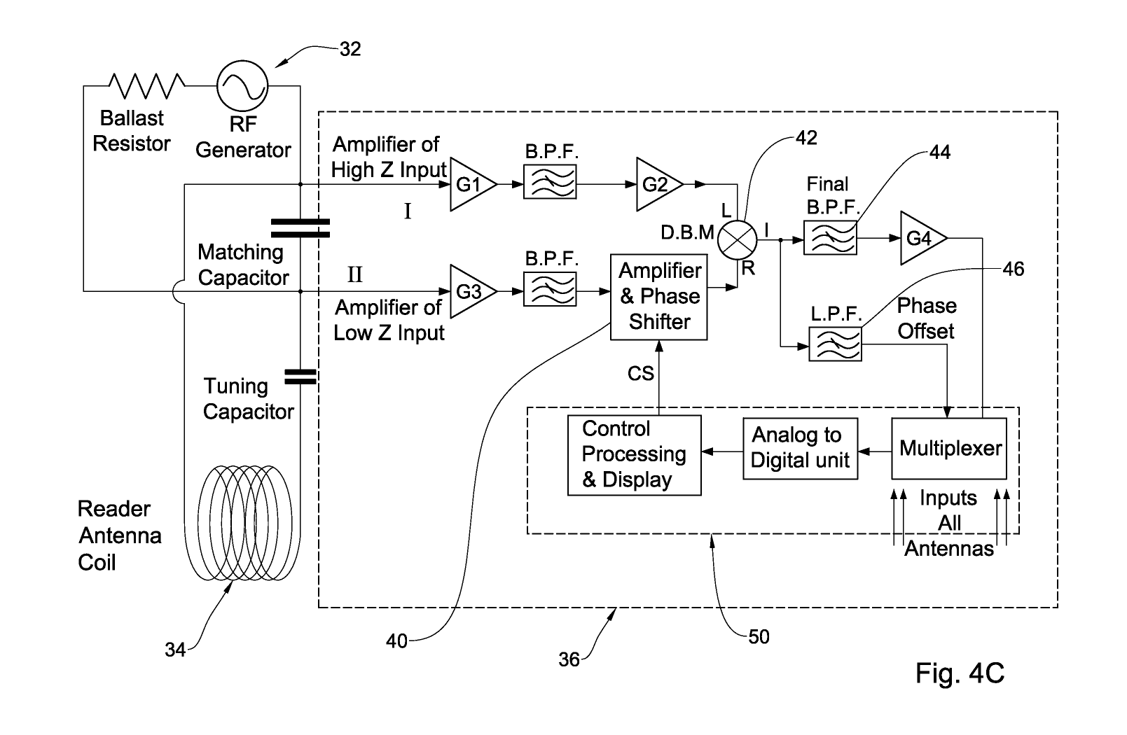

In the examples of FIGS. 4A and 4B, the high RF electromagnetic field source and sensor 32,34 is configured for powering (exciting) and detection through a series matching capacitor antenna circuit. In the example of FIG. 4C, the unit 32 is configured for powering and detection through a parallel matching capacitor antenna circuit.

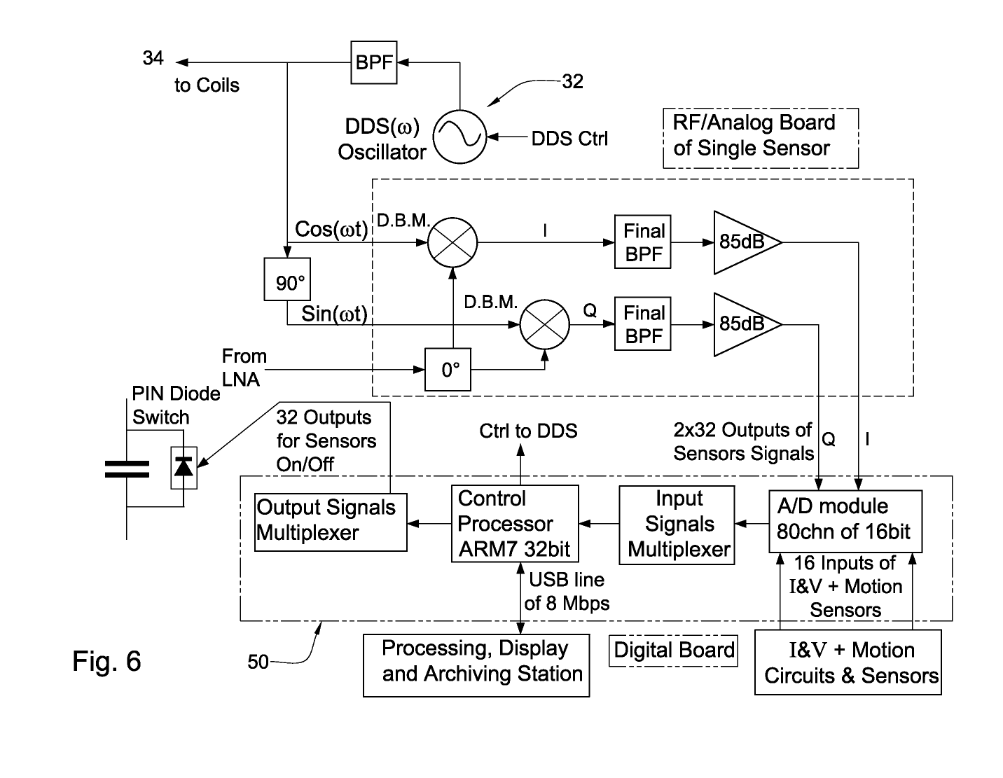

In all the examples, the receiver unit 36 is configured as an appropriate hardware and software utility that analyzes the inputs from all the sensing coils. In some embodiments of the invention, e.g. as shown in FIGS. 4A-4C, the receiver unit 36 includes two signal channels I and II, channel I for a reference signal (i.e. the powering signal generated by the source 32), channel II for a received signal (electromagnetic signal of a carrier frequency of the detection signal generator modulated by the field distortion induced by the marker). The reference channel I includes a so-called “buffer” amplifier G1 of a high-impedance input and preferably also a frequency filter B.P.F. (operating around the power signal frequency, e.g. 125 KHz) to filter out the generator induced noise, and optionally includes a further amplifier G2. The channel II includes a low impedance input amplifier G3 and its associated frequency filter B.P.F. operating around the power signal frequency, e.g. 125 KHz. Also provided in the channel II is an amplifier and phase shifter 40. The phase shifting might be needed to facilitate further processing of the signals coming from these two channels and also for the calibration purposes. As will be exemplified further below with reference to FIGS. 6, 7, 9 and 12, a need for the phase shifting and calibration procedures can be eliminated. The signals from the two channels I and II are received at a mixer 42 which may be configured as Double Balanced Mixer (D.B.M.). The mixing applies signal multiplication, and the resulted output is divided into two parts: one part passes through a frequency low pass filter 46 , and the other part passes through a frequency band pass filter 44 operating around 1-32 Hz and then through a further buffer amplifier G4.

In the example of FIG. 4A, a rectifying diode unit 43 is used for envelope detection at the D.B.M. output of one part of the multiplied signal that passes through the rectifier unit 43 and then through the frequency low pass filter 46 which operate together to extract the DC component from the multiplied signal. It should be noted that the DC component yields a maximum DC level, if the phase shift between the two input channels I and II that input to the D.B.M. were one time adjusted to 0°. Therefore, in this example, the powering and detection of the distortion pattern produced by the location marker is performed through a series matching capacitor antenna circuit and a rectifying diode for adjusting the phase shift at the D.B.M. input. FIG. 4B illustrates another configuration of the electrical circuit for detecting minimum DC level without a rectifier unit. In this example, the DC component yields a minimum DC level if the phase shift between the two input channels I and II to the D.B.M. were one time adjusted to 90°.

As described above, the D.B.M. output is divided into two parts, one towards the frequency low pass filter 46 (e.g. via rectifying diode 43 as in FIG. 4A), and the other towards the frequency band pass filter 44 and buffer amplifier G4. These separated signal parts enter a multiplexer of a processor unit 50, which in a similar way receives such separated signal parts from each one of the sensing coils, and is configured for further processing these signals to determine the marker location. The processor unit 50 thus includes a multiplexer and analog-to-digital converter, and a data processing and analyzing utility. The processor unit 50 may include other utilities, such as memory, display, etc. The data processing and analyzing may be configured for applying autocorrelation function to the coil-associated signal associated with the predetermined periodic low-frequency modulation (stored in the memory). The processor unit 50 also includes a calibration utility for generating a control signal CS to the buffer amplifier and phase shifter 40.

Thus, the detection system senses the marker through its mismatched in return power received through the amplifier G4 at the low marker gated frequency, while the phase shift between the two input channels I and II (inputs to G1 and G3) are one time, prior to the processing, adjusted to 0° as with the electrical circuit of FIG. 4A and to 90° as with the electrical circuit of FIG. 4B. It should be noted that when the phase shift between the two input channels I and II to the D.B.M. is adjusted to 90°, the detected marker modulation signal that input to the frequency band pass filter 44 is around zero DC level and thus the dynamic range of the detected information is enhanced. The following is an example of the analysis of phase shift optimization between channels I and II to the input of the D.B.M. multiplier.

The reference signals, S1 and S3, sampled through amplifiers G1 and G3 respectively, are as follows.)

S1(0°)=Sin(ωt)—for a zero phase difference with signal S3;)

S1(90°)=Cos(ωt)—for 90° phase difference with signal S3;

S3=[1+a·Sin(Δωt)]·Sin(ωt)=Sin(ωt)+a·Sin(Δωt)·Sin(ωt)

where a<1 is the peak modulation amplitude at frequency of Δω, and a·Sin(Δωt)·Sin(ωt)=(a/2)·[Cos(ωt−Δωt)+Cos(ωt+Δωt)];.

Therefore:

S3=Sin(ωt)+(a/2)·[Cos(ωt−Δωt)+Cos(ωt+ωt)]

Accordingly, the mixer D.B.M. yields at its output the following products:

1. When the phase difference between S1 and S3 is 0°:

S1·S3=Sin(ωt)·{Sin(ωt)+(a/2)·[Cos(ωt−Δωt)+Cos(ωt+Δωt)]}=Sin(ωt)·Sin(ωt)+(a/2)·Sin(ωt)·Cos(ωt−Δωt)+(a/2)·Sin(ωt)·Cos(ωt+Δωt);

Sin(ωt)·Sin(ωt)=(½)−(½)·Cos(2ωt);

Sin(ωt)·Cos(ωt−Δωt)=(½)·[Sin(2ωt−Δωt)+Sin(Δωt)];

Sin(ωt)·Cos(ωt+Δωt)=(½)·[Sin(2ωt+Δωt)+Sin(Δωt)];

S1·S3=a·Sin(Δωt)+(½)+(½)·Cos(2ωt)+(a/2)·[Sin(2ωt−Δωt)+Sin(2ωt+Δωt)].

Here, only the modulation term, a·Sin(Δωt), remains through the frequency band pass filter 44, while the DC level that input to the frequency low pass filter 46 is equal to (1+2a) with the use of a rectifier diode (FIG. 4A) and is equal to (½) without the use of a rectifier diode (FIG. 4B). Thus the phase adjustment of 0° yields maximum input DC level to the low pass filter 46.

2. When the phase difference between S1 and S3 is 90°:

S1·S3=Cos(ωt)·{Sin(ωt)+(a/2)·[Cos(ωt−Δωt)+Cos(ωt+Δωt)]}=Cos(ωt)·Sin(ωt)+(a/2)·Cos(ωt)·Cos(ωt−Δωt)+(a/2)·Cos(ωt)·Cos(ωt+Δωt) Cos(ωt)·Sin(ωt)=(½)·Sin(2ωt);

Cos(ωt)·Cos(ωt−Δωt)=(½)·[Cos(2ωt−Δωt)+Cos(Δωt)]

Cos(ωt)·Cos(ωt+Δωt)=(½)·[Cos(2ωt+Δωt)−Cos(Δωt)];

S1·S3=(½)·Sin(2ωt)+(a/2)·[Cos(2ωt−Δωt)+Cos(2ωt+Δωt)].

Here, accordingly there is no modulation term that remains through the frequency band pass filter 44, while the DC level input to the frequency low pass filter 46 is equal to (½+2a) with the rectifier diode and is equal to 0 without the rectifier diode at optimum and will allow higher dynamic range for the detected signal.

FIG. 4C exemplifies the electrical circuit of the detection system in which the powering and detection of the distortion pattern produced by the location marker is performed through a parallel matching capacitor antenna circuit including a ballast resistor and without a rectifying diode for adjusting the phase shift at the D.B.M. input. In this specific example, lower running voltages can be used in the antenna resonance circuit relative to ground.

The complex impedance with the parallel matching capacitor setup can be calculated as follows:

1/Z=jωCₘ+1/[R_{S}+jωL+1/(jωCₜ]=jωCₘ+1/{R_{S}+j[(ωL−1/(ωCₜ)]}

where Cₘ and Cₜ are the matching and tuning capacitors respectively; R_{S} is the Ohmic impedance of the loop antenna that stems from the skin effect loss in the coil antenna wire and from added radiation loss due to the periodical loading by the switching marker when brought in the range of the antenna electromagnetic field.

1/Z=R_{S}/{R_{S}²+[(ωL−1/(ωCₜ)]²}+j{ωCₘ−[(ωL−1/(ωCₜ)]/{R_{S}²+[(ωL−1/(ωCₜ)]²}}

Re(1/Z)=R_{S}/{R_{S}²+[(ωL−1/(ωCₜ)]^{2]}}

Im(1/Z)=ωCₘ−[(ωL−1/(ωCₜ)]/{R_{S}²+[(ωL−1/(ωCₜ)]²}

C tan(φ)=[Im(1/Z)]/[Re(1/Z)]

where (φ) is the phase angle between the voltage and current across the matching capacitor.

The matching and tuning capacitors of the antenna circuit transform the internal R_{S} to pure 50Ω across the matching capacitor as for example in the RF generator terminals driving constant RF current with the help of a series ballast resistor, typically of 50Ω too, implies:

Re(1/Z)=1/50, i.e. 1/50=R_{S}/{R_{S}²+[(ωL−1/(ωCₜ)]²}

Im(1/Z)=0, i.e. 0=ωCₘ−[(ωL−1/(ωCₜ)]/{[R_{S}²+[(ωL−1/(ωCₜ)]²}

The periodical loading by the switching marker causing change in the Ohmic impedance of the loop antenna, induces changes in the phase angle (φ) as well as in the measured RF voltage across the matching capacitor recorded by amplifier G3. Practically, lower running voltages can be used in powering the antenna resonance circuit with parallel matching capacitor.

It should be noted that the autocorrelation function enables to extract information when the detected signal is periodic and of a predictable shape. The capability for incorporating autocorrelation processing to the final received signal even more enhances the final obtained signal to noise ratio.

The enhancement in signal to noise ratio by the system of the present invention enables to increase the detection distance. Taking into accounts, the magnetic field strength decays with 1/r³, when r is the distance from the center of a loop antenna, every 10 dB of relative increase in the signal to noise ratio is equivalent to increase by a factor of approximately 1.5 in the maximal detection range.

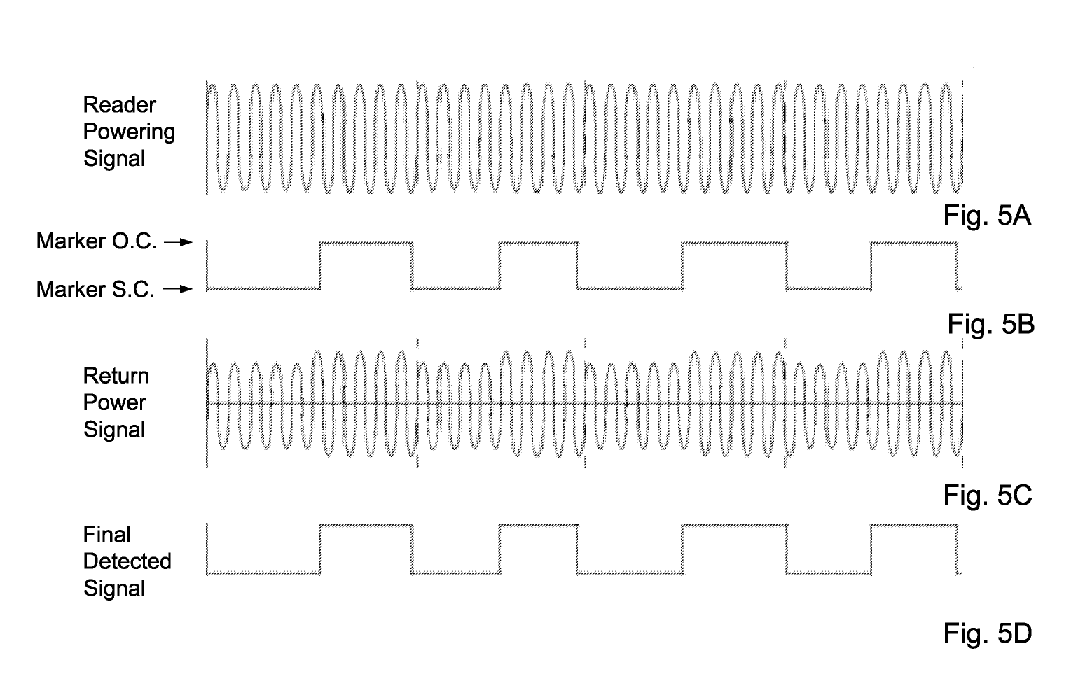

Reference is made to FIGS. 5A-5D exemplifying the signals generated and received by the above described detection system: FIG. 5A shows a continuous powering signal generated by the system sampled through high input Z buffer amplifier Gl. FIG. 5B shows a distortion pattern B₂ (shown in FIGS. 1A-1B) produced by the location marker of the present invention, Marker O.C corresponding to the ON switch mode (i.e. open circuit state of the electrical circuit within the marker) while S.C corresponding to the OFF switching mode (i.e. short circuit state of the electrical circuit within the marker). FIG. 5C shows a return power signal sampled through low input Z amplifier G3 in which the reader powering signal (of FIG. 5A) carriers the distortion pattern B₂ (of FIG. 5B). FIG. 5D shows the final detected signal as sampled through the high input Z amplifier G4.

The following are some more examples of the configuration of the receiver unit to be used in the detection system.

FIG. 6 shows the receiver unit configuration utilizing the quadrature approach of two analog sampled channels. The reference signal is generated by a Direct Digital Synthesizer (D.D.S.), and a Band-Pass Filter (B.P.F) operates to filter out noise coming from the D.D.S. In the quadrature approach configuration, the reference signal generated by the D.D.S. is divided in two parts associated with two channels, the incident (I) and the quadrature (Q) channels. In the Q channel, the reference signal is shifted by 90° relatively to the I channel and is multiplied by a sine wave signal. The signal in the I channel is multiplied by a cosine wave signal. The detection system senses an external magnetic source (e.g. a marker) through its mismatched in return power received through the Low Noise Amplifier (L.N.A.) at the external source gated frequency, while the phase shift between the two input channels (reference and input to the L.N.A.) are one time, prior to the processing, adjusted to 0°. The signals in the I and Q channels pass through a Final Band-Pass Filter (B.P.F) operable to filter out the generator induced noise. The signals in both channels are then amplified by amplifiers designated as 85 dB. The RF oscillator frequency generated from a Direct Digital Synthesizer (D.D.S.) may be for example of high frequency of 134.2 KHz, and the Final Band Pass filters are designed for flat response and may be for example of frequencies in the range of about e.g. 32 Hz to 2.2 KHz. The Multipliers (D.B.M.) are implemented for example with AD734, and the Amplifiers (‘85 dB’) having a gain in the range of e.g. 60-90 dB may be implemented with e.g. AD797. The amplitude imbalance between the I and Q channels can be calibrated through the system software.

This quadrature detection circuit is advantageous in signal reception as it involves down conversion to baseband (i.e. eliminating the need of a DC offset) through mixing the reference signal and with the reference signal shifted at 90 degrees from the reference. The ratio between the signal and the noise is substantially completely utilized, leading to a gain of 3 dB in the signal to noise ratio, and in addition out of band noise rejection is achieved .The use of I-Q channel configuration eliminates a need for using a phase shifting unit.

The return power may be received by an 85 dB gain amplifier. The powering and detection system contains multiplicity of RF coils (in this specific example 32 coils which activate the marker(s) and senses the gated marker(s) through mismatch in the return power as emerged eventually through the 85 dB gain amplifier. The receiver incorporates mixing of the return mismatch in antenna power that sampled through L.N.A. amplifier, having low input Z at the carrier frequency of the charging electromagnetic field, to yield the complex output information at a low gated frequency which thus minimizes noise through the FINAL low received bandwidth that equals to the marker gated frequency, thus yielding the maximum theoretically possible signal-to-noise ratio.

Moreover, the electrical circuit includes a PIN diode switch connected to the phase coils' arrays configured and operable to select the coils involved in the detection process. For example, if certain coils are located in the surrounding of a noisy source, their contribution to the detection process may be eliminated in real-time by operating the PIN diode switch.

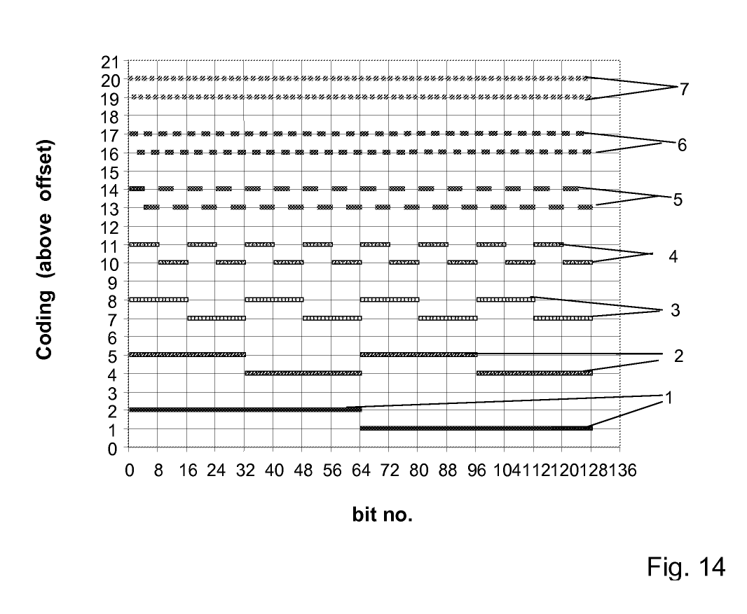

It should be noted that the recorded markers signal having square waves configuration as illustrated in FIG. 5D, includes only radix-2 frequencies, namely 2ⁿ Hz where n=4 to 10 in integer increment. The square wave function may be synchronized to Cos(Δωt) or Sin(Δωt), depending if the function is sampled Symmetrical or Asymmetrical around time t=0. Quadrature sampling after multiplication of the carrier modulated with information by Cos(Δωt) or Sin(Δωt) respectively, creating the Incident (I) and Quadrature (Q) channels, alleviating inherently the necessity for phase correction, is implemented for sampling. As illustrated in FIG. 14 all harmonics are Odd (1, 3, 5, . . . ) and have the same multiplication constant (4/π), the marker's location could be determined uniquely from the fundamental frequency (Δω), where no collision whatsoever between the marker frequency of Even values only could occur with the harmonics which are all Odd.

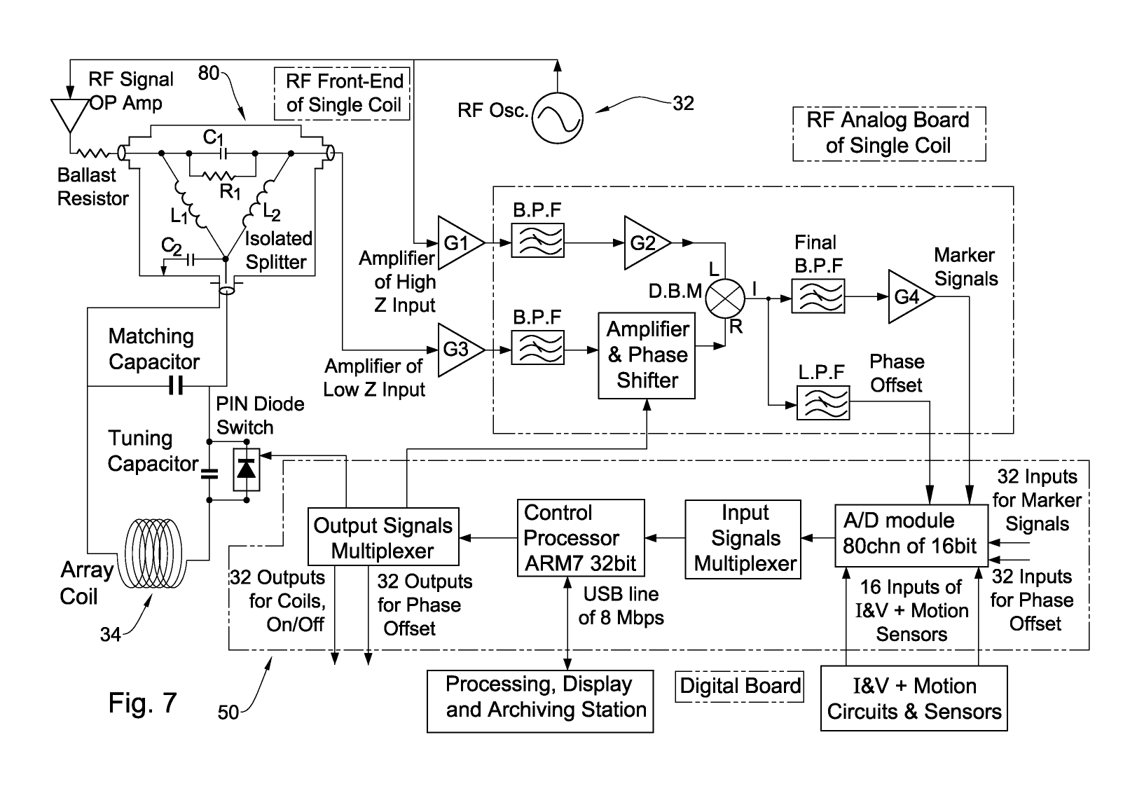

FIG. 7 shows an example, of the receiver unit configuration utilizing an isolated splitter unit 80. The use of the isolated splitter unit is aimed at filtering out the electromagnetic component of the powering stage that would otherwise be received back by the coils and need to be further processed. Thus, with the isolated splitter unit, data to be processed is free of the powering field component. In the configuration illustrated in FIG. 7, the D.D.S. generator is replaced by other kind of appropriate RF Oscillator. The RF Analog Board of a Single Coil is identical to the one of FIG. 4A.

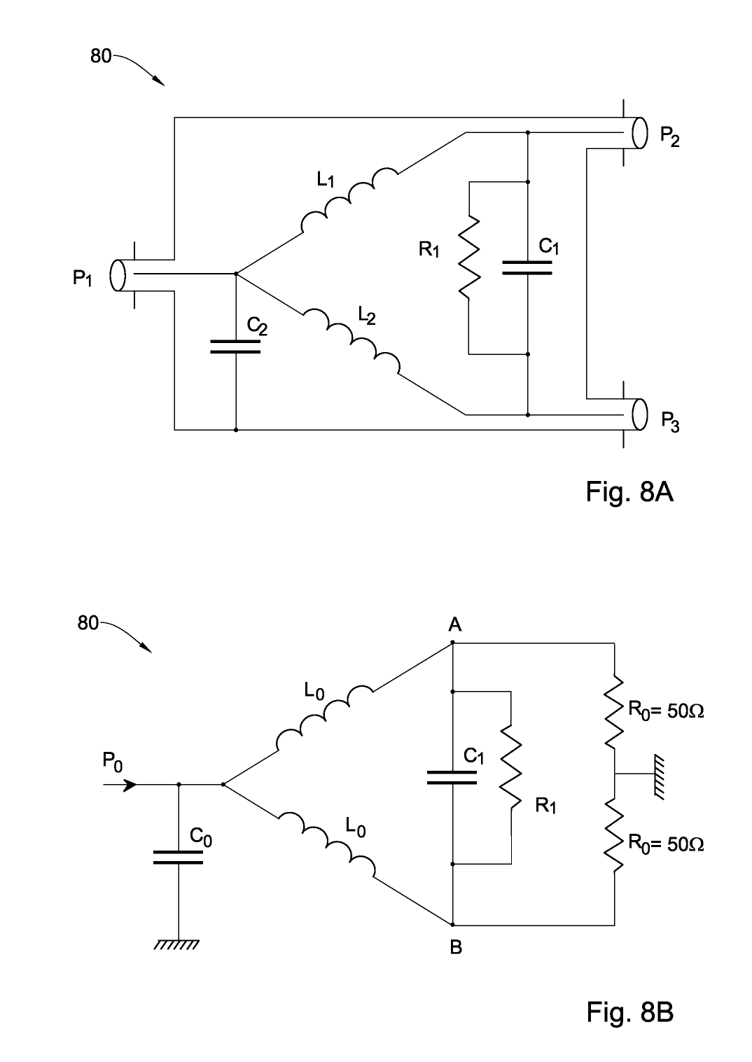

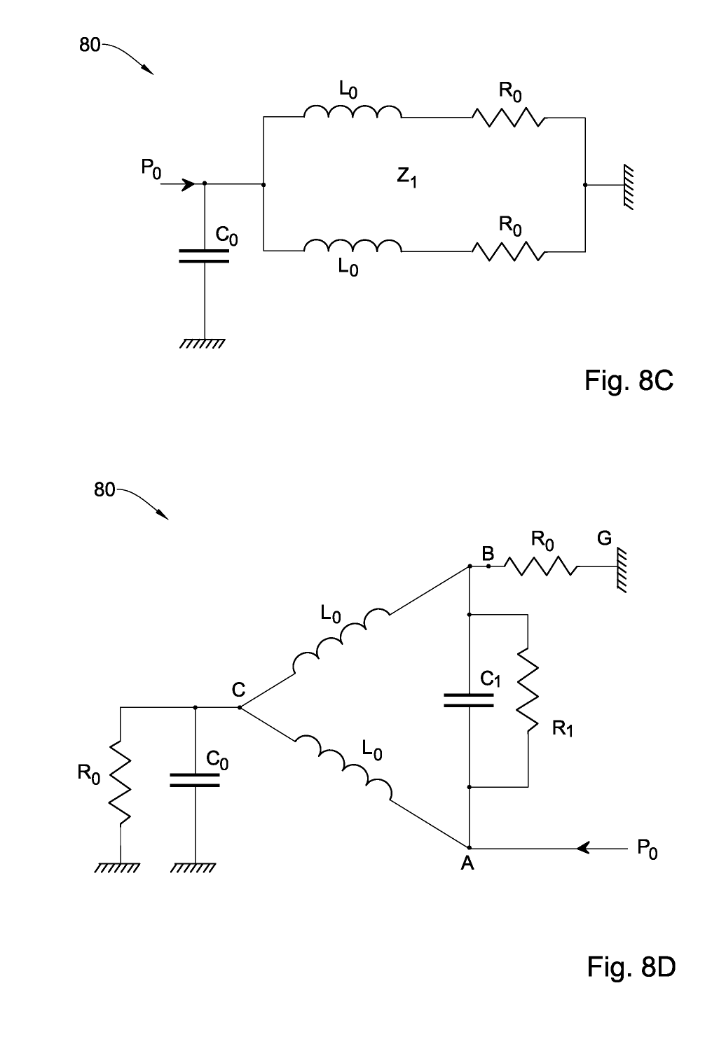

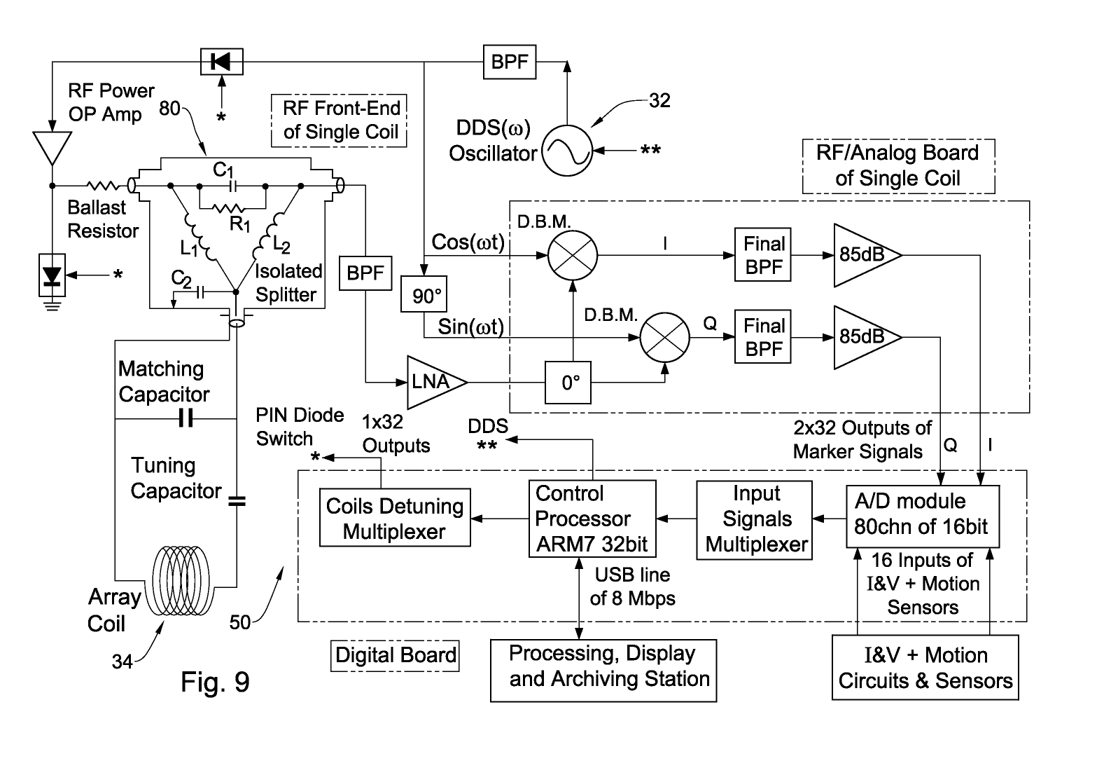

In this connection, reference is first made to FIGS. 8A-8D illustrating an isolated splitter electrical circuit, configured to match impedance of 50Ω at each port. As shown the isolated splitter circuit has three ports P₁-P₃, ports P₁ and P₂ for connecting to the receiving coil and the RF power amplifier, and port P₃ for connecting to the receiver unit. The amplifier of the receiver unit (connected to port P₃) thus senses only the coil's mismatch in return power due to the gated marker.

The values of the elements in the isolated splitter unit have to fulfill the following conditions:

R1=100Ω; L1=L2=50/ω; C2=1/(50ω); C1=1/(100ω)

where ω=2πf and f is the RF frequency of the signal.

In the example of FIG. 8B, the values of the matching capacitor C₀ and of the splitting conductors L₀ can be calculated as follows:

$$\frac{1}{{Z}_{1}} = \frac{2}{jω {L}_{0} + {R}_{0}}$$

$$\frac{1}{Z} = jω {C}_{0} + \frac{1}{{Z}_{1}} = jω {C}_{0} + \frac{2}{jω {L}_{0} + {R}_{0}} = jω {C}_{0} + \frac{2 ( {R}_{0} - jω {L}_{0} )}{{R}_{0}^{2} + {( ω {L}_{0} )}^{2}}$$

$$\frac{1}{Z} = jω {C}_{0} - \frac{2 jω {L}_{0}}{{R}_{0}^{2} + {( ω {L}_{0} )}^{2}} + \frac{2 {R}_{0}}{{R}_{0}^{2} + {( ω {L}_{0} )}^{2}} = \frac{1}{50}$$

$${R}_{0} + \frac{{( ω {L}_{0} )}^{2}}{{R}_{0}} = 100 ;$$

$${R}_{0} = 50 ;$$

$$ω {L}_{0} = 50 Ω ;$$

$$ω {C}_{0} = \frac{1}{50} Ω or \frac{1}{ω {C}_{0}} = 50 Ω$$

FIG. 8C corresponds to the equivalent electrical circuit of FIG. 8B where there is an exact power splitting V_{AB}=0.

FIG. 8D correspond to the equivalent circuit where good isolation is provided with V_{BG}=0 wherein B is also a grounded point. Here, the dissipating resistor R₁ and the balancing capacitor C₁ across R₁ can be calculated as follow:

$$\frac{1}{{Z}_{AB}} = j {wc}_{1} + \frac{1}{{R}_{1}} = \frac{1 + j {wc}_{1} {R}_{1}}{{R}_{1}}$$

$${Z}_{AB} = \frac{{R}_{1}}{1 + jω {C}_{1} {R}_{1}} = {R}_{1 *} \frac{1 - jω {C}_{1} {R}_{1}}{1 + {( ω {C}_{1} {R}_{1} )}^{2}}$$

$${Z}_{A C} = jω {L}_{0}$$

$$\frac{1}{{Z}_{CB}} = \frac{1}{jω {L}_{0}} + \frac{1}{{R}_{0}} + jω {C}_{0} = \frac{1}{{R}_{0}} + j ( ω {C}_{0} - \frac{1}{ω {L}_{0}} )$$

$${Z}_{CB} = {R}_{0}$$

$${Z}_{A C} + {Z}_{CB} = jω {L}_{0} + {R}_{0} = {R}_{0} ( 1 + j ) = {Z}_{ACB}$$

$$IF ω {C}_{1} {R}_{1} = 1 ;$$

$${Z}_{AD} = {R}_{1} = ( \frac{1 - j}{2} ) and if {R}_{1} = 100 Ω$$

$$then \frac{1}{ω {C}_{1}} = 100 Ω and {Z}_{AB =} {R}_{0} ( 1 - j )$$

Therefore, the signal power P₀ from B is divided equally between Z_{AB} and Z_{ACB} and the power is dissipated on Z_{BG}.

FIG. 9 illustrates the quadrature approach as illustrated in FIG. 6 combined with the use of an isolated splitter unit 80.

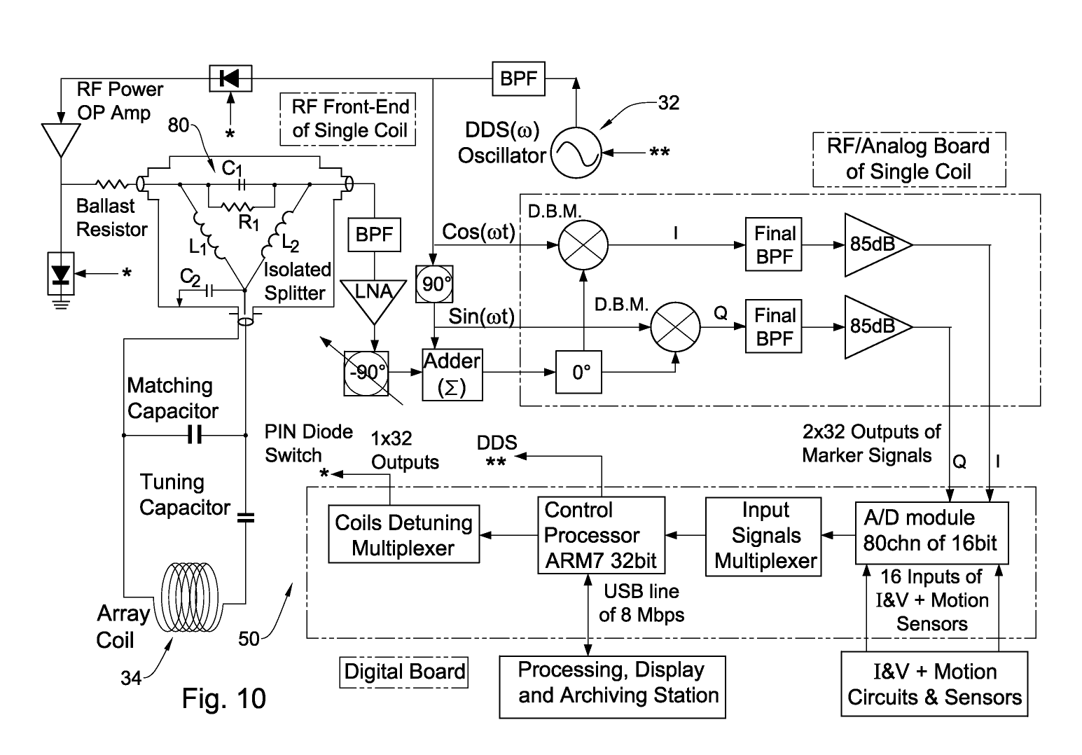

In the configuration of FIG. 10, a phase cancellation is performed between the reference signal and the marker signal. The marker signal is filtered out by a B.P.F. and amplified through a L.N.A. Then, the signal is shifted by a −90°. An adder element sums the reference signal shifted by a 90° and the marker signal shifted by a −90°. The resulting signal is therefore only the variation of the marker signal without the “carrier signal” of the magnetic field generated by the phase coils' array. In this configuration, the pre-calibration and the phase adjustment are thus eliminated. In the example of FIG. 10, the use of the Isolated Splitter design at the feed point of each coil, together with the subtraction of the residual non isolated carrier by Phase Cancellation with the −90° shift and Adder (E) element, allow high input RF power to the array coil through the matching capacitor while keeping the L.N.A. Amplifier of Low Z Input, isolated from the RF Power Amplifier to yield enhanced dynamic range for the detected baseband signal by the I&Q multipliers.

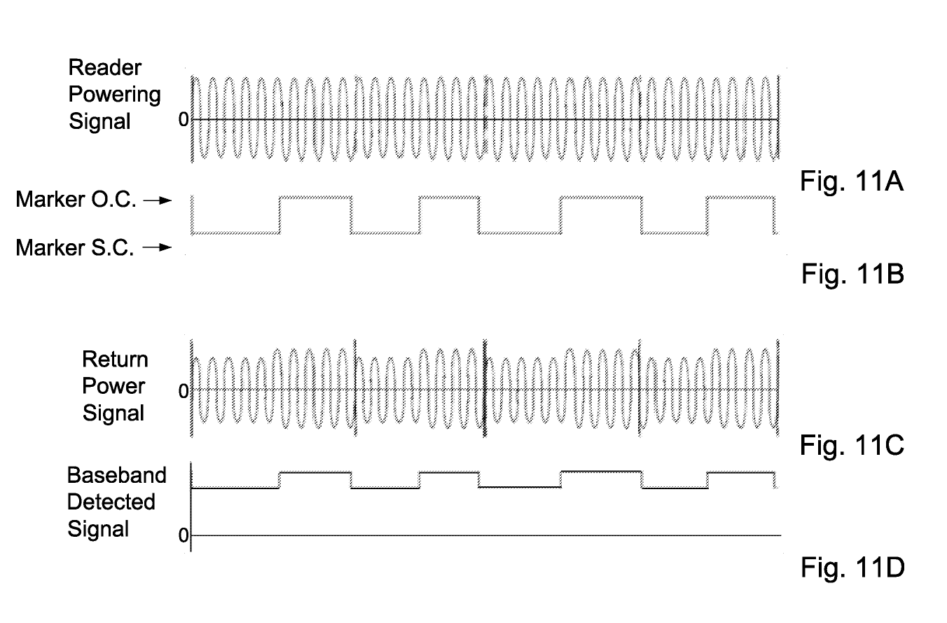

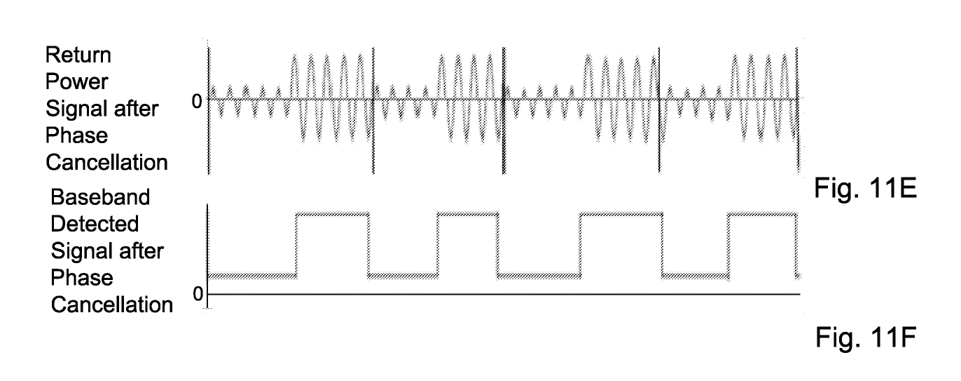

Reference is made to FIGS. 11A-11F illustrating the Timing diagram describing from top to bottom the continuous ‘Reader Powering Signal’ (FIG. 11A), the activated marker switched between ON/OFF Marker states (FIG. 11B). Also shown is the Return Power Signal of mismatch on the coil's matching capacitor as sensed by the L.N.A. Amplifier (FIG. 11C). FIG. 11D illustrates the baseband detected signal in the quadrature channel before the Final Band Pass Filter in the configuration of FIG. 9. FIG. 11E illustrates the return power signal after the phase cancellation process in the configuration of FIG. 10. FIG. 11F illustrates the baseband detected signal in the quadrature channel before the Final Band Pass Filter after the Phase cancellation in which the dynamic range has increased in comparison with the baseband detected signal of FIG. 11D.

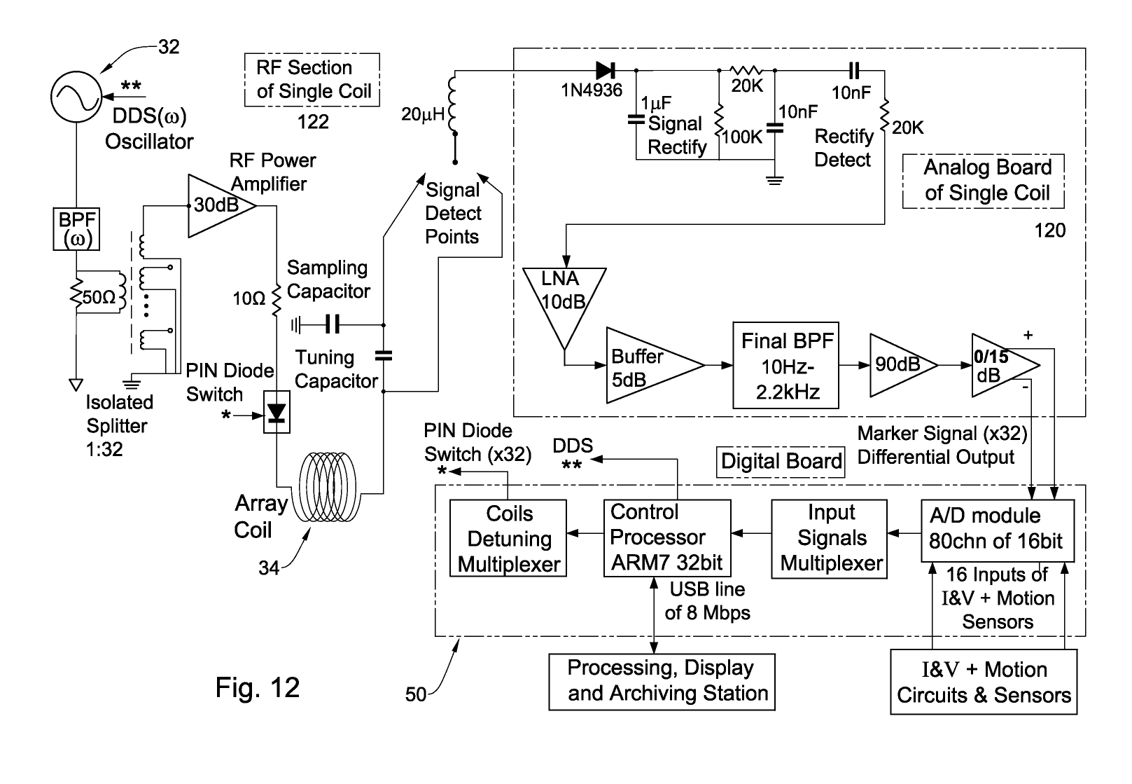

FIG. 12 illustrates yet another possible configuration of the receiver unit implemented in the Analog board of a single sensor module 120 wherein a series resonance circuit replaces the parallel matching capacitor circuit of the precedent FIGS. 6, 7, 9, 10 with the use of signal rectify and rectify detect circuit elements. Here, the input reference RF signal to the power amplifier of each coil is distributed from a central D.D.S. frequency source through a transformer with one primary and multiple secondary windings (in this specific example 32) associated with the multiple coils respectively. As an alternative, multiple resistances in parallel may also divided the input reference signal into 32 components. Thus, the multiplicity of RF coils activate the marker(s) and senses the gated marker(s) through sensing the resulted modulated current on the loaded coil by the marker through the signal rectify and rectify detect circuit elements that emerged eventually through gain amplifier of 105 to 120 dB gain at baseband frequency. The resistance designated 10Ω is a protection resistance against shorted load. It should be noted that in this configuration, the signal is directly detected on the circuit and not through reference channels or according to the quadrature approach. Therefore, at the signal detect point, the signal is high and has a high voltage (for example 100V). The signal rectify and the rectify detect elements transform the high input signal into a high signal having a small voltage (for example 0.01V). The DC of the signal of the rectify detect element does not pass through the capacitor (designated 10 nF), and only the modulation of the signal indicative of the marker location enters the L.N.A.

Moreover, as illustrated in the RF section of a single coil module 122, the electrical circuit may include a sampling capacitor configured to divide the voltage and to reduce the high voltage level of the signal. The working voltage value may be therefore reduced by using a sampling capacitor however, the signal to noise ratio would be decreased.

The output information is provided at a low gated frequency minimizing the noise through the FINAL low received bandwidth that equal to the marker gated frequency to yield enhanced dynamic range for the detected baseband signal.

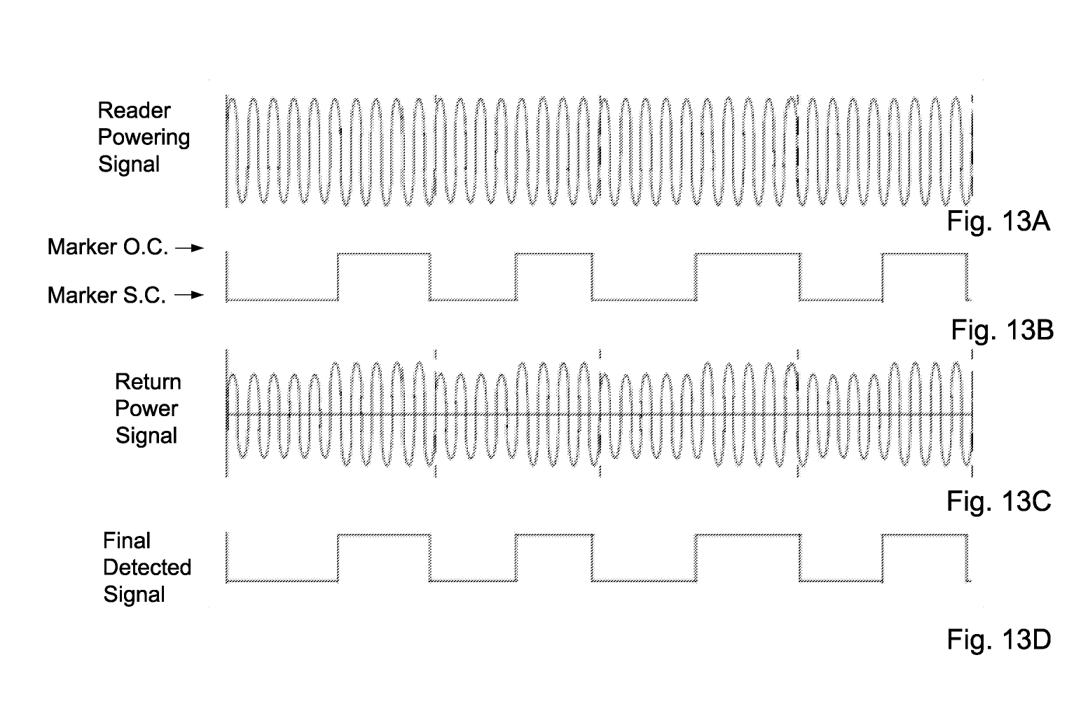

FIG. 13A-13D shows the timing diagram of signals obtainable with the receiver unit configuration of FIG. 12. The diagram shows from top to bottom the continuous ‘Reader Powering Signal’, the activated marker that switched between ON/OFF states of connect ‘Marker S.C.’ (Short Circuit) to disconnect ‘Marker O.C.’ (Open Circuit)(FIG. 13A). Also shown are the Rectified Signal due to the gated Marker at the low gated frequency, e.g. 32 Hz, on the Sampling Capacitor (FIG. 13B) and the Baseband Detected Signal out of the ‘Signal Rectify’ and Rectify Detect’ that entered to the L.N.A. (FIG. 13C), which followed by the Final Band Pass Filter of low bandwidth that help in increasing the signal to noise ratio (FIG. 13D).

It should be understood that the target location system of the present invention may be used for locating multiple markers, e.g. associated with multiple locations in the subject or patient. It should be noted that for obtaining accurate 3D information on the location of the treated anatomy two or preferably three implanted markers are required. In such a case, the teachings herein can easily be extended to multiple markers. Each powered marker is gated in turn at different low gated frequency and the multiple markers are detected/located simultaneously.

In this connection, reference is made to FIG. 14 illustrating the coding of seven markers as a function of bit number. In this example, the programmed markers are of 128 (=2⁷) bit at RF frequency of 131,072 (=2¹⁷) Hz and encapsulated inside glass enclosure of e.g. 2 mm diameter and 13 mm length. The code contains for example seven different identities, each highlighted in graphical form in which Marker #n, having an even effective bit rates per second of 2ⁿ, where n=5 to 11 is in integer increment.

It should be noted that the principles of the present invention for the target location by powering and detecting by the same antenna arrays can advantageously be used for locating a target in a patient's body. The technique allows placing the target location system below the patient being monitored/treated, e.g. configuring the target location system as a layer within the patient's support. For example, the system of the present invention may be incorporated within a patient's bed being above the conventionally used stretcher. It should be understood that typically the system is installed in the ceiling of the treatment room at about the mid distance between the patient's bed and the ceiling. The bed may be driven by the operator in the control room to maneuver at various in/out, up/down and rotation positions during the radiation process.

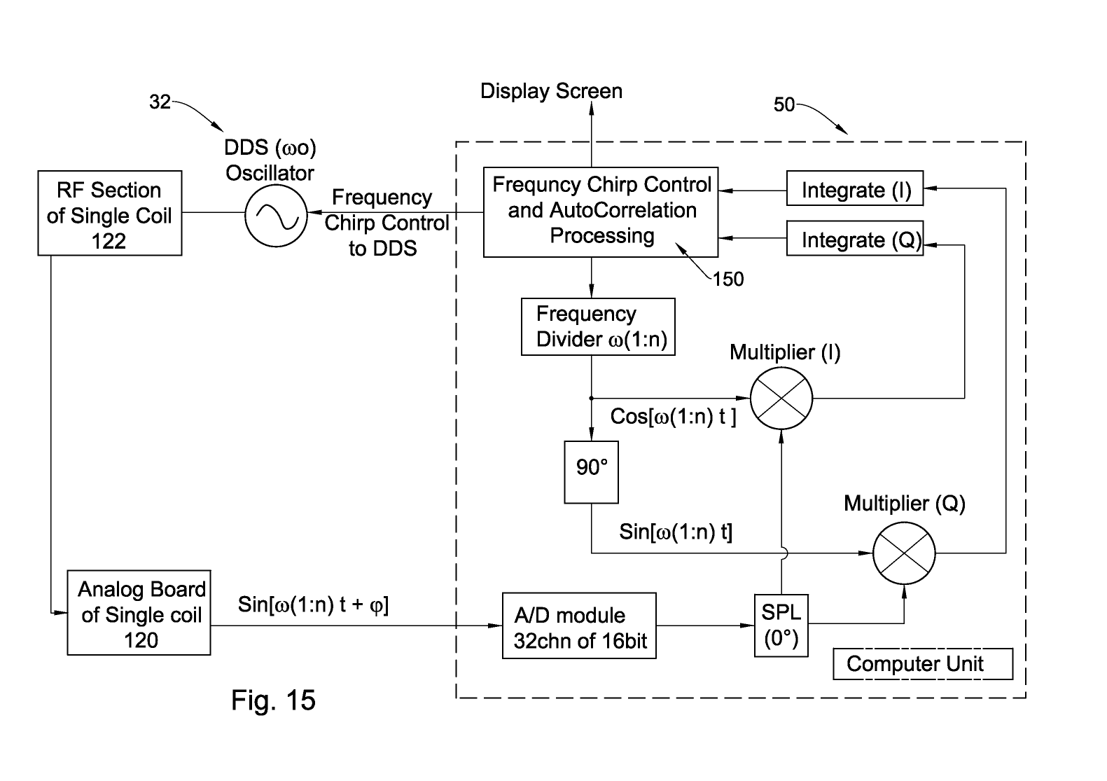

Reference is made to FIG. 15 illustrating an example of an electrical circuit for extracting the frequency component of the marker. Here, the functional parts of the detection system are implemented by an analog board of a single coil 120 and an RF section of single coil 122 similarly to the configuration described in FIG. 12. In this specific example, the powering and detection system contains a multiplicity of RF coils (in this specific example 32 coils), and activates the marker(s) and senses the gated marker(s) through mismatch in the return power as emerged eventually through a gain amplifier. In this example the software utility or processing unit 50 includes a frequency chirp control and autocorrelation processing utility 150. The reference signal generated by the D.D.S. 32 is divided by a frequency divider in two parts associated with two channels, the incident (I) and the quadrature (Q) channels. The frequency chirp control and autocorrelation processing utility 150 auto-correlates (i.e. multiplies and integrates) the received marker signal with the Q and I references from the D.D.S. in synchronization with the marker signals, thus yielding to a free interference signal strength enabling an accurate determination of the markers location. Using this interference rejection approach, unwanted input is rejected.

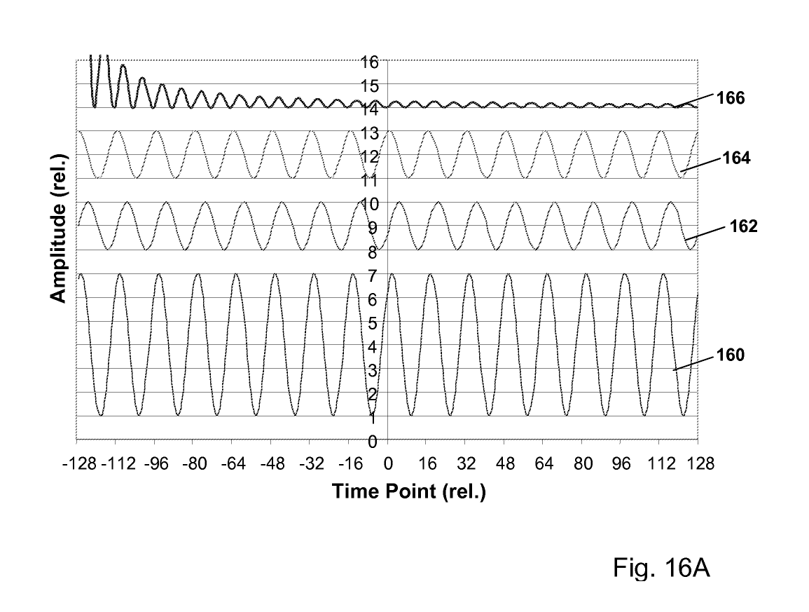

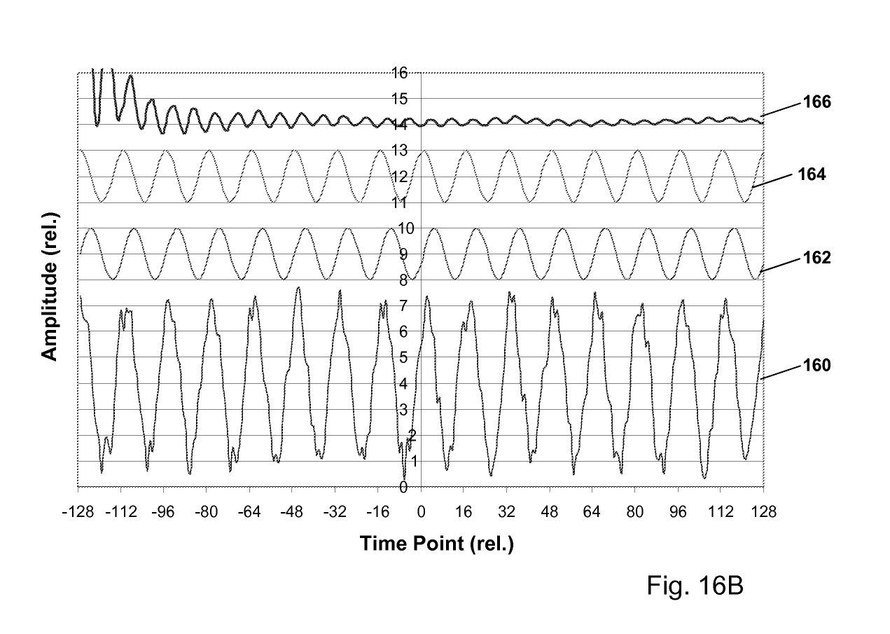

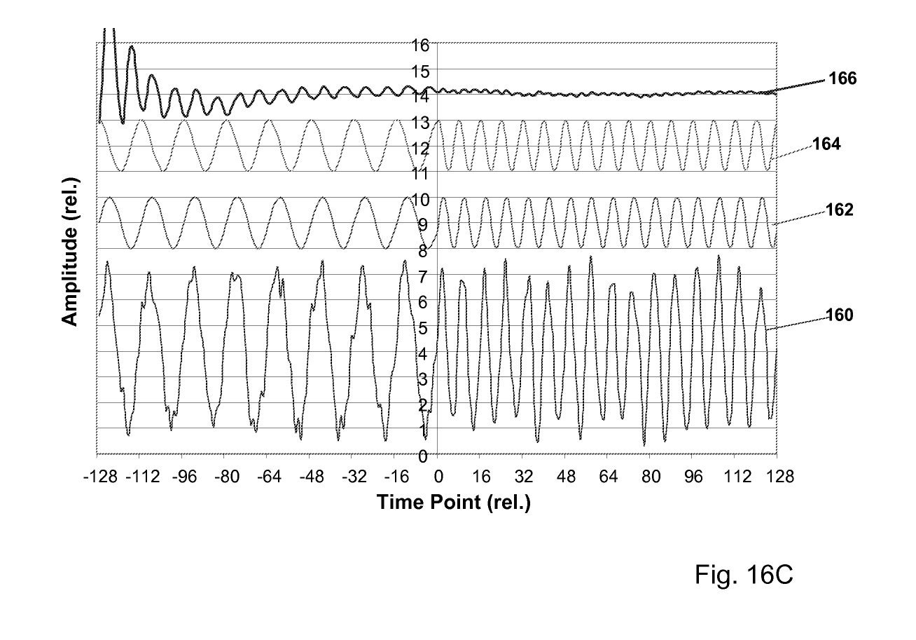

Reference is made to FIG. 16A exemplifying simulated signals generated and received by the detection system of FIG. 15. For the sake of comparison, a synthetic simulated marker signal 160 is autocorrelated, namely multiplied and integrated with the chirped I reference 162 and Q reference 164 from the D.D.S. in frequency synchronization with the received Marker signals. The autocorrelation result is represented in 166. In FIG. 16B, the received marker signal 160 having some noise components, is autocorrelated and synchronized similarly to the process illustrated in FIG. 16A. As illustrated in 166, the autocorrelation result is similar to the results received for the “pure” synthetic marker signal of FIG. 16A. Therefore, the autocorrelation process eliminates noise components. In FIG. 16C, a noisy signal is received from the RF coils at two different chirped frequencies 160 and autocorrelated, namely multiplied and integrated at two different frequencies with the chirped I reference 162 and Q reference 164 from the D.D.S. in frequency synchronization with the received Marker signal. As in FIG. 16B, the autocorrelation process enables the elimination of the noise components.

It should be noted that, as disclosed above, the antenna arrangement may include three arrays of sensing coils, each defining a signal detection (e.g. receiving) aperture, where the arrays are located in three non-parallel (preferably orthogonal) planes, respectively. The detection apertures (coils) of each array are arranged in a spaced-apart relationship along a respective one of the three planes.

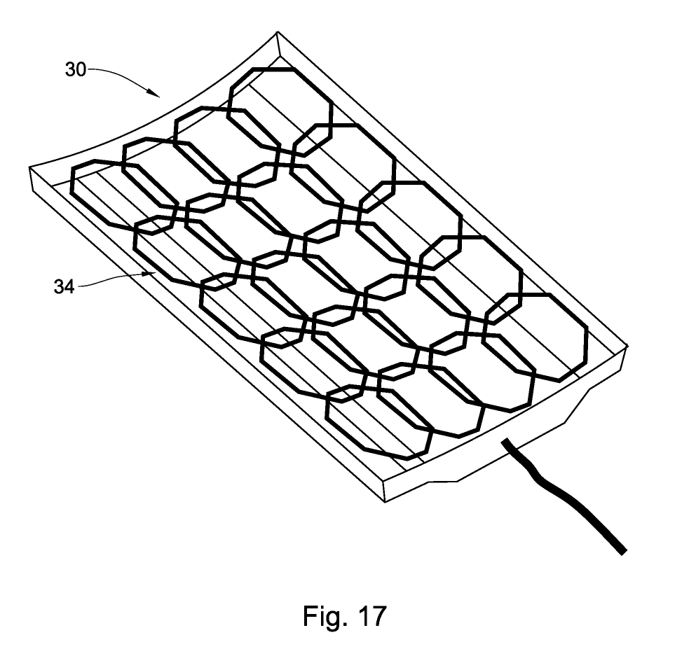

As illustrated in FIG. 17, each of the sensing coils within the array of coils 34 is positioned so as to have substantially no interaction with all adjacent coils. The sensing coils are configured and arranged such that the apertures of locally adjacent coils located in each plane are overlapping partially, to set their mutual inductance to zero, thereby eliminating the problem of splitting resonances due to inductive coupling with nearest neighbors. Also, interactions between non-adjacent coils are minimized by coupling each onto an associated preamplifier having relatively low input impedance (e.g. 5 Ohm). Each coil is connected to the input of an associated one of a like plurality of low-input-impedance preamplifiers, which minimize the interaction between any coil and other coils not immediately adjacent thereto. Interactions between non-adjacent coils are minimized by coupling each onto the associated preamplifier.

As indicated above, the detection system of the present invention comprises three arrays of coils, each for detecting a direction to the marker (source of magnetic field) according to one of three non-parallel axes. This is implemented, for example, by arranging three arrays of coils in three orthogonal planes.

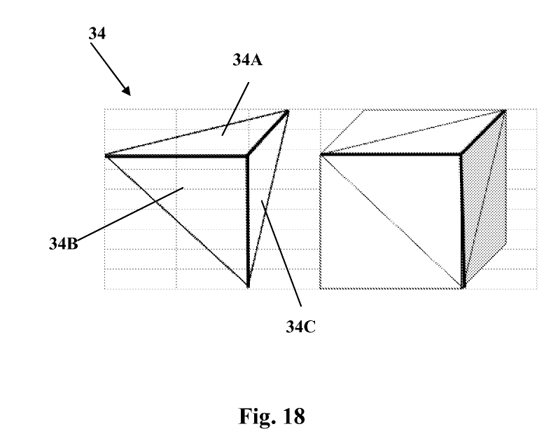

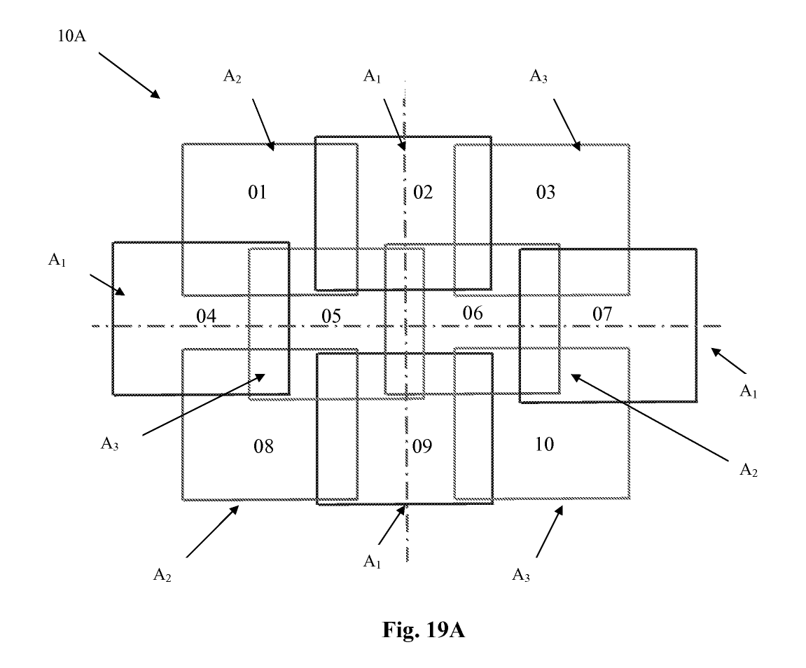

Reference is made to FIG. 18 representing an isometric view of the antenna arrangement 34. This arrangement defines three sensing apertures 34A, 34B and 34C along three orthogonal planes, each sensing aperture being constituted by a respective array of coils (array of sensing apertures). More specifically, on each surface multiple coils (circular coils) are placed for minimized coupling of any two adjacent coils in the array, such that the apertures of locally adjacent coils located in each plane are overlapping partially, to set their mutual inductance to zero, thereby eliminating the problem of splitting resonances due to inductive coupling with nearest neighbors

The local magnetic field created by the marker generates distortions of the magnetic field of the antenna arrangement, which distortions are detected by different sensing coils as the so-called “loading responses”. Output signals from the coils are different as corresponding to different coordinates of detection (different coils' locations) within a volume defined by the arrays. Each different response loading is used to construct a different one of a like plurality of samples, which are then combined at the processor unit 50, on a point-by-point basis, to produce a single composite load of a total sample portion from which response loading contribution was received by any of the array of coils. After the receiver unit has completed the signal processing, the resulting output of the receiver digital output loadings may then be used by the processor unit 50 to locate the marker. Each digital output loading is a measurement of one component of the magnetic field integrated over the aperture of the sensor array. The detection system determines the location of the marker (i.e. marker location) from a set or array of measurements taken from the sensors (i.e. set of actual measurements). The reading volume delimits the three-dimensional area in which the marker can be localized. As three arrays are used associated with three non-parallel planes, the marker location is determined more accurately than with single coils array as an intersection of three directions determined by three arrays, respectively. This enables real-time determination of 3D location of a marker accurately without a need for using any reference data about loading responses associated with known locations. The marker of the present invention may for example be configured as a disposable cylindrical package having dimensions of about 12 mm length by less than 2 mm diameter, preferably 9.5 mm length by 1.8 mm diameter. Actually the dimensions of the marker are defined by the required dimensions of the coil-on-core unit to generate, by the external field, an electric current of a magnitude capable of energizing the switch.

The marker velocity through reading volume may be up to approximately 5 meters per second. The detection system is stationary and the marker moves through a fixed reading volume. A plurality of markers may be present in the reading volume simultaneously. It should be noted that the same detection system can be used for locating multiple markers. In some applications, multiple markers are associated with the same subject. Multiple markers in the detection system field may be activated simultaneously. Each marker may carry a chip configured with a different low gated frequency and can thus be distinguished from other marker, while all being located sequentially.

It should be noted that marker optimization (size vs. performance) is a principle issue in the marker design. For a given marker technology, a marker with a larger coil or with ferrite core of higher permeability can be activated in a lower field strength and can give a longer reading distance. For a given marker volume (diameter times length), the amount of the volume occupied by the coil on ferrite core assembly should be maximized.

In the “low-frequency” domain (low switching frequency) utilized by the marker of the present invention, energizing frequencies between 100 KHz and 150 KHz can be used. The detection system continuously generates an AC magnetic field at a constant field value (constant peak amplitude), and the marker produces a modulated load while energized by said magnetic field.

The antenna system includes a receiving antenna arrangement, which is configured with a reduced cross-talk between the receiving antennas, and also to enable determination of the 3D location of a marker. The receiving antenna arrangement is formed by three (or more) phase arrays of closed-loop coils, which have substantially the same polygonal (for example square) geometry and substantially the same surface area (e.g. 20×20 cm). The three antenna arrays are located in three substantially parallel planes, respectively, and are arranged such that each coil of one array is superimposed along at least two sides thereof by coils from at least one other array with certain polygonal overlapping regions. The latter are substantially small as compared to the polygonal antenna surface.

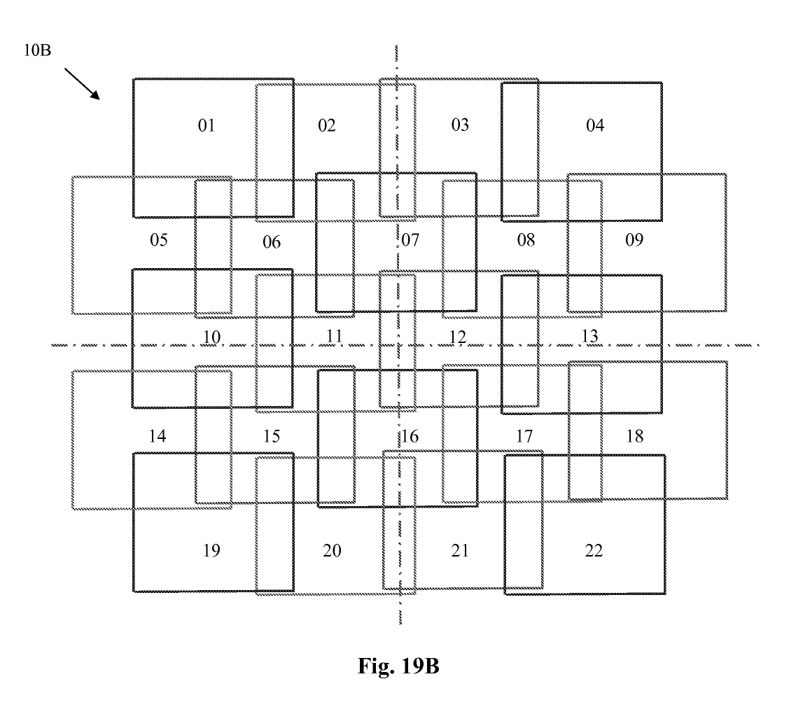

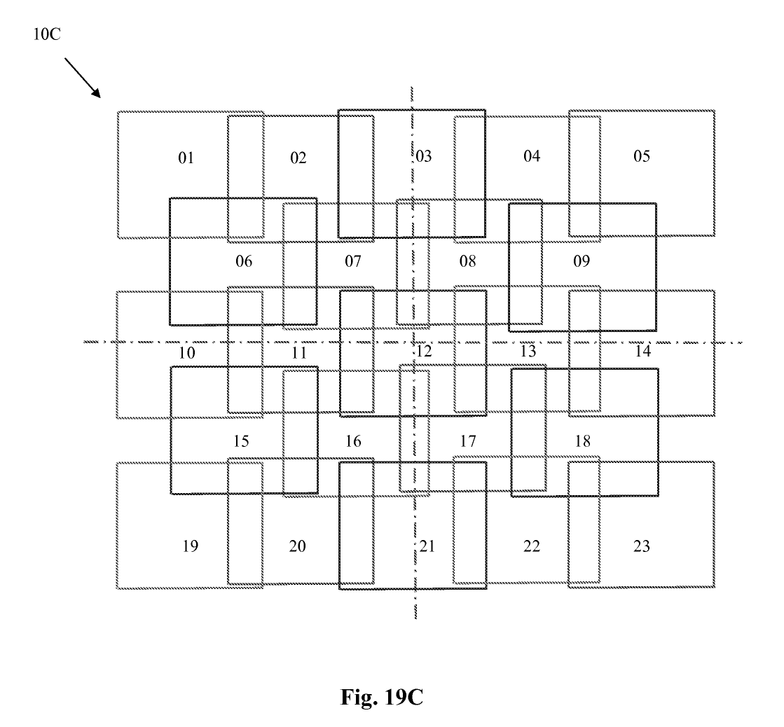

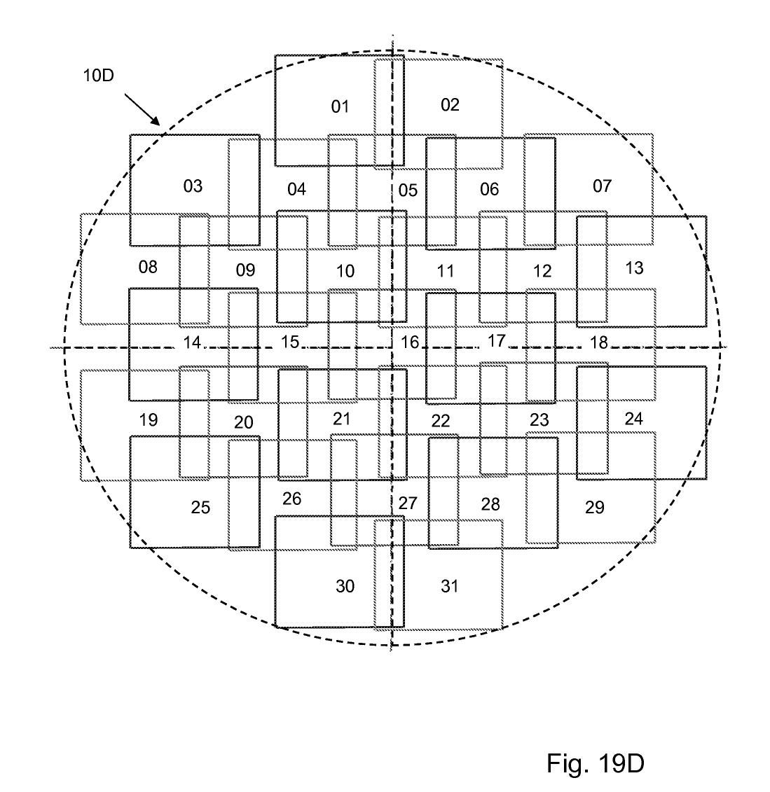

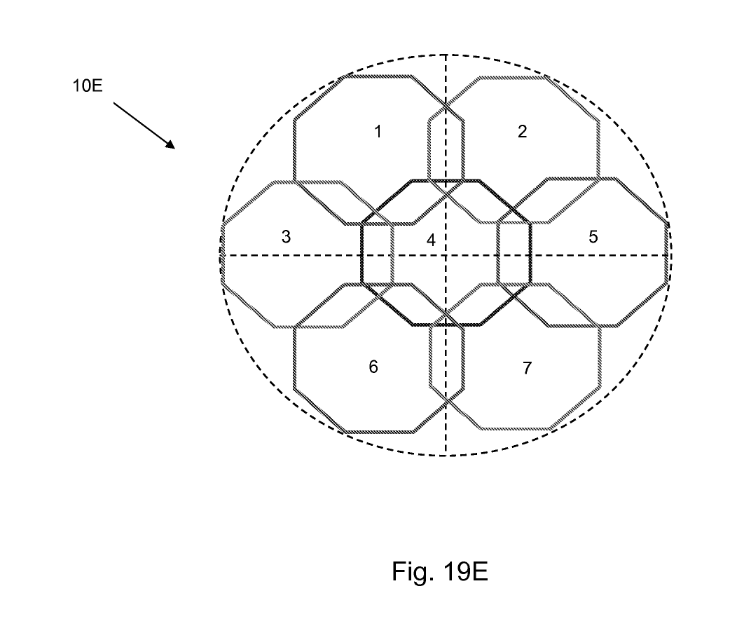

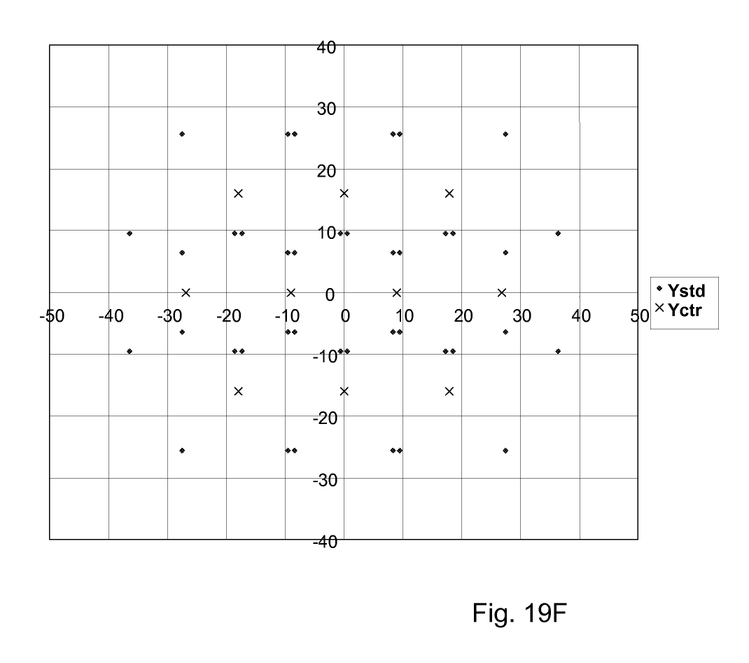

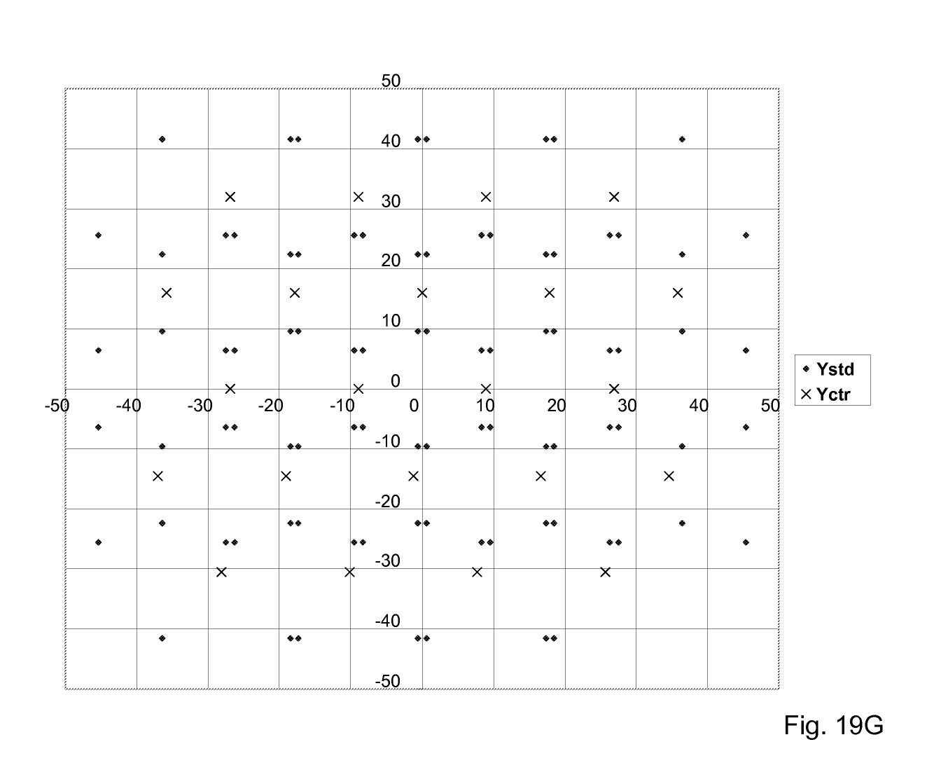

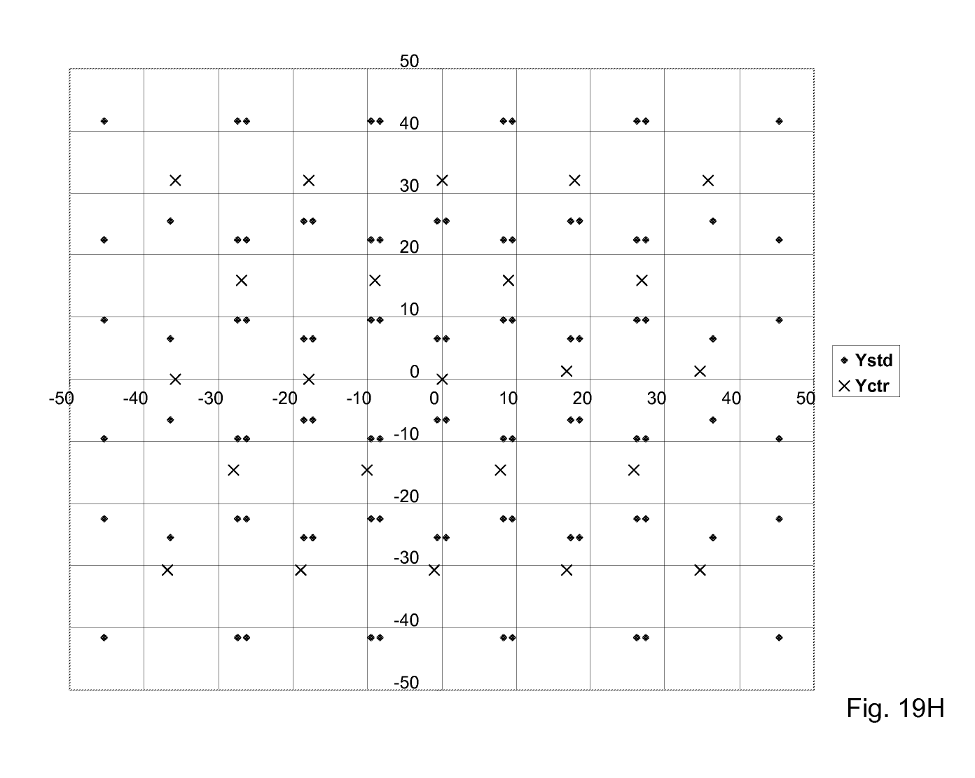

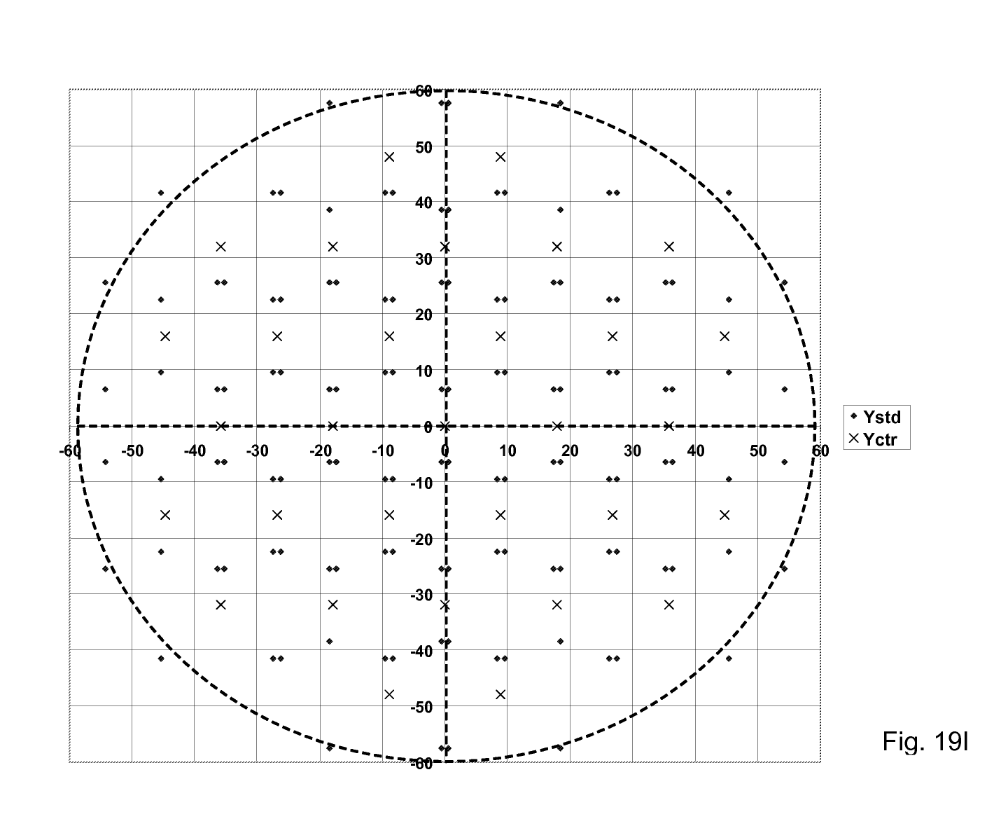

The following are some examples of the arrangement of receiving antennas. FIG. 19A shows antenna arrangement 10A, including ten 20×20 cm coils, 01, 02, . . . , 10, arranged in three arrays A₁, A₂ and A₃ located in three parallel spaced-apart planes, respectively, of the total size of 75 cm width and 55 cm length. FIG. 19B shows antenna arrangement 10B, including twenty two 20×20 cm coils, 01, 02, . . . , 22, arranged in three arrays A₁, A₂ and A₃ (not shown) located in three parallel spaced-apart planes, respectively, of the total size of 90×90 cm. FIG. 19C shows antenna arrangement 10C including twenty three square coils, 01, 02, . . . , 23, arranged in three arrays A₁, A₂ and A₃, (not shown) of the total size 90×90 cm. FIG. 19D shows antenna arrangement 10D including thirty one square coils, 01, 02, . . . , 31, arranged in three arrays A₁, A₂ and A₃ (not shown), of the total size 110×110 cm. FIG. 19E shows antenna arrangement 10E, including seven octagon loops of 40 cm width arranged in three arrays A₁, A₂ and A₃ (not shown) located in three parallel spaced-apart planes, respectively. The arrangement is contoured by a circle having a diameter of 120 cm.

It should be understood that generally any polygonal geometry, preferably symmetrical, can be used. The higher number of facets in the polygon, the better the filling factor and larger space regions covered by non-overlapping antenna coils are achievable by the coil arrays. Thus, this last configuration (19E) enables a better filling factor and larger regions covered by non-overlapping antenna coils in comparison with the rectangular configuration (for example 19D), that altogether result in more efficient energy trapping for the received marker signal.

It should also be understood that the fact that the marker of the present invention has a low-frequency response to the external field, allows for the marker detection with a smaller field of view by the receiving antenna system. Accordingly, a smaller number of coils (per detection surface) may be used.

Each antenna array is a two-dimensional array, i.e. coils of each array are arranged in first and second plurality of rows along first and second perpendicular axes, respectively. Each coil of one antenna array is along at least two sides thereof superimposed by two coils of the two other antenna arrays, respectively, defining two overlapping regions.

For example, as shown in FIG. 19A, coil 02 of array A₁ at its opposite sides is superimposed by coils 01 and 03 of arrays A₂ and A₃ respectively; and coil 09 of array A₁ at its opposite sides is superimposed by coils 08 and 10 of arrays A₂ and A₃ respectively. Coil 01 of array A₂ is at one side thereof superimposed with coil 02 of array A₁ and at the other adjacent side partially superimposed by coil 04 of array A₁ and partially superimposed by coil 05 of array A₃.

The coils of each antenna array are arranged with a certain space between them, such that each antenna array has at least one pair of coils arranged with the two coils of the other two arrays, respectively, being aligned with said space. For example, as seen in FIG. 19A, coils 05 and 06 of arrays A₃ and A₂ are located in the overlapping fashion between coils 04 and 07 of antenna array A₁; coils 06 and 07 of arrays A₂ and A₁ are located in the overlapping fashion between coils 03 and 10 of antenna array A₃.

In some embodiments, the arrangement is such that coils of each antenna array are arranged in first and second groups of perpendicular rows, such that a number of coils in the row is different by 1 from a number of coils in the locally adjacent parallel row. This is exemplified in FIGS. 19A, 19B and 19C with respect to both horizontal and vertical rows, and in FIG. 19D with respect to vertical rows. The arrangement may be such that each of the extreme (uppermost and lowermost or left and right) rows of one group of rows has n antennas, and each of the intermediate parallel rows has (n+1) antennas. This is a horizontal rows arrangement of FIG. 19D.

The coils' arrangement may be such that a central region of the arrangement falls on the overlapping region between the coils, as shown in the examples of FIGS. 19A, 19B and 19D; or such that the coil region is located at the center as in the example of FIG. 19C.

It should be understood that electrical currents flowing in two parallel sides of two coils defining an overlapping region compensate magnetic fields created by said current. By this a cross talk between the adjacent antennas is reduced, and even more reduced in case these two overlapping coils are located in spaced-apart planes. This is illustrated in FIGS. 19F-19I corresponding to the coils' arrangement of FIGS. 19A-19D, respectively. As shown, each coil “does not see” the others, and is represented by its four corner points, thus allowing measurement of the polar coordinates of an external magnetic field (or modulated pattern) source with high signal-to-noise.

The following are theoretical considerations for calculating the magnetic field produced by phase array coils comprising rectangular loops of wire:

Let us consider static and time varying fields that are quasi-static. In the latter case, the wavelength λ of the time varying electromagnetic field is much greater than any dimension or distance of interest. For example, a 100 KHz alternating field (λ≅3,000 m) is well approximated as being quasi-static a few meters or less from loops of comparable dimensions.

The quasi-static condition allows solving the static field problem and, with negligible error that introduces the time dependence as a multiplicative factor, the direct current in the field equations could be replaced with an alternating current. The field equations are for rectangular loops with a single turn of wire. The magnetic flux density for loops with more than one turn is found by multiplying the equations by the appropriate number of turns [Martin Misakian: Equations for the magnetic field produced by one or more rectangular loops of wire in the same plane. J. Res. Natl. Inst. Stand. Technol. 105, 557-564 (2000)].

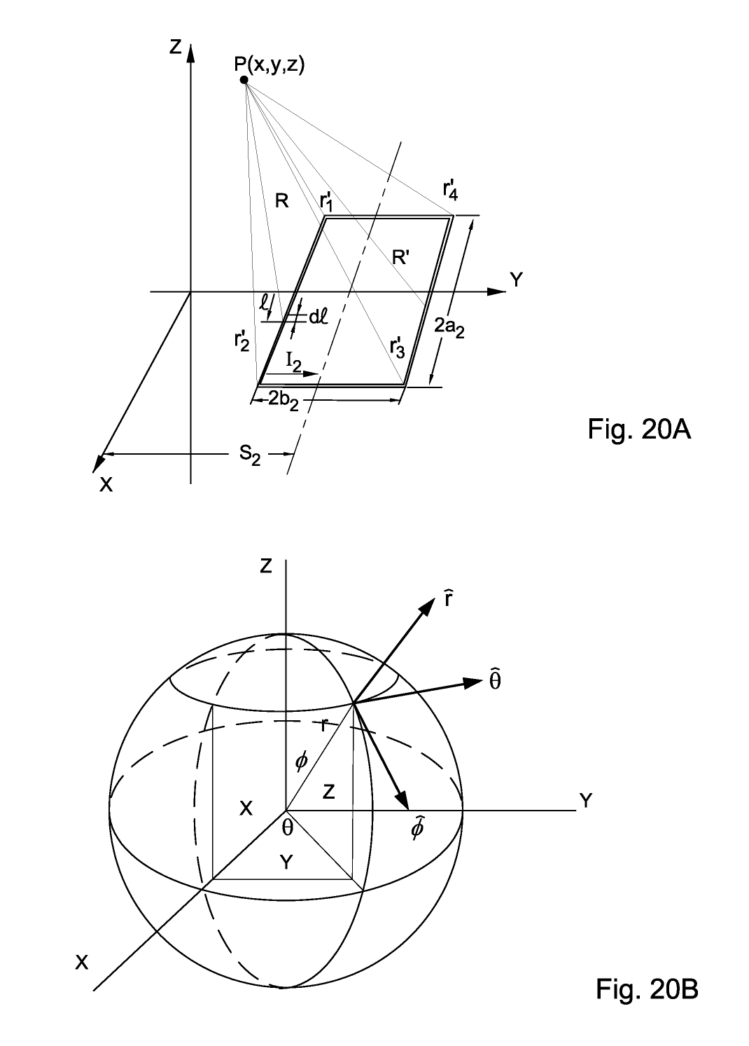

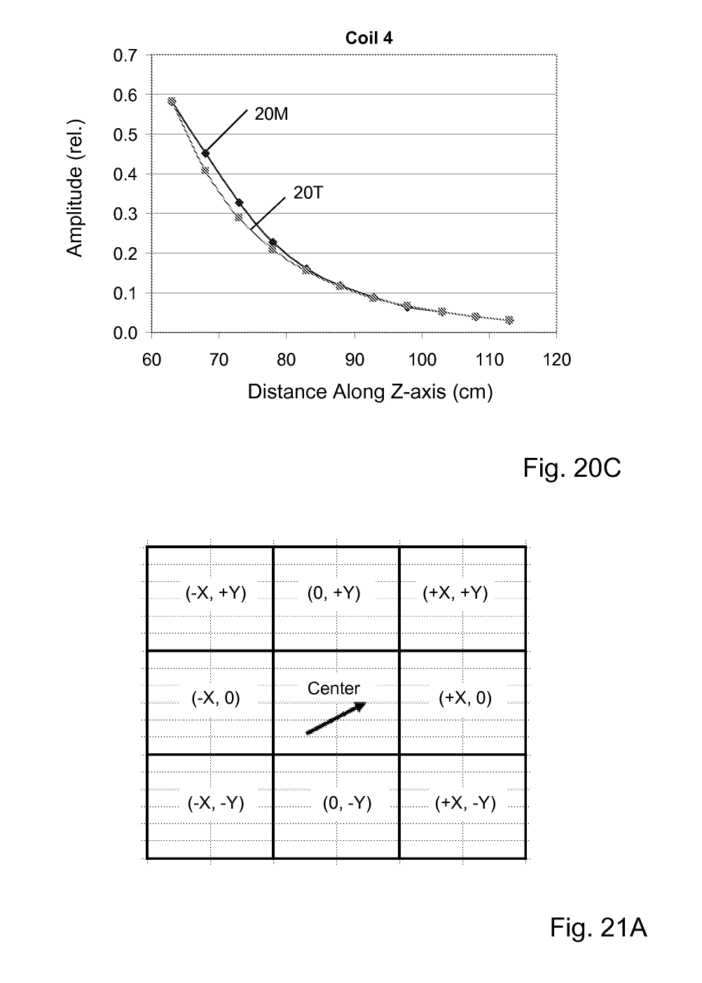

As shown in FIG. 20A, for a rectangular loop of wire in the x-y plane of negligible wire cross section, with side dimensions 2a₂ by 2b₂, the parameters r₁, r₂, r₃, and r₄ are the distances from the corners of the loop to point P(x, y, z) where the magnetic flux density is evaluated (i.e. point P is considered as the marker location). In this calculations the magnetic flux propagation in vacuum (μ₀ being the magnetic permeability of vacuum) is considered.

For direct current I₂ in the loop, the direction of the magnetic flux density will remain fixed and is described by the vector given below:

B=Bₓ₁i+B_{y1}j+B_{z1}k,

where i, j, and k are unit vectors along the x, y, and z directions, respectively.

The magnitude of the magnetic flux density vector will be also constant and equal to

P(x,y,z)=|B|=(B²ₓ₁+B²_{y1}+B²_{z1})^{1/2},

For a given array of coils, a plurality of P(x,y,z) values is measured. It should be noted that by using three arrays of coils located in spaced-apart parallel planes, the pointing error is reduced because more data points are taken into account in the calculated average result, and the Standard Deviation is minimized. Moreover, the powering of the marker could be achieved at a higher distance using three dimensional phase array coils powered by the electrical current flowing in the same direction (namely clockwise or counter-clockwise) and in the same phase along time.

Coordinates Sₓ and S_{y} (represented by S₂ in the figure for the square coil loop) are the coils’ center coordinates along x and y axes respectively.

The analytic derivation yields the following expressions in sequence for the generated magnetic field by the quasi static electrical current in each corner of the square coil:

The z-component of the magnetic flux density P(x,y,z) is:

$${B}_{z 1} = \frac{{μ}_{0} {I}_{1}}{4 π} {\sum }_{a = 1}^{4} [ \frac{{( - 1 )}^{α} {d}_{α}}{{r}_{α} [ {r}_{α} + {( - 1 )}^{α + 1} {C}_{α} ]} - \frac{{C}_{α}}{{r}_{α} [ {r}_{α} + {d}_{α} ]} ] , \\ where$$

$$\begin{matrix}{C}_{1} = - {C}_{4} = {a}_{1} + x & {d}_{1} = {d}_{2} = y + {b}_{1} \\ {C}_{2} = - {C}_{3} = {a}_{1} - x & {d}_{3} = {d}_{4} = y - {b}_{1}\end{matrix}$$

$${r}_{1} = \sqrt{{( {a}_{1} + x )}^{2} + {( y + {b}_{1} )}^{2} + {z}^{2}}$$

$${r}_{2} = \sqrt{{( {a}_{1} - x )}^{2} + {( y + {b}_{1} )}^{2} + {z}^{2}}$$

$${r}_{3} = \sqrt{{( {a}_{1} - x )}^{2} + {( y - {b}_{1} )}^{2} + {z}^{2}}$$

$${r}_{4} = \sqrt{{( {a}_{1} + x )}^{2} + {( y - {b}_{1} )}^{2} + {z}^{2}} . \\ {B}_{x 1} = \frac{{μ}_{0} {I}_{1}}{4 π} {\sum }_{α = 1}^{4} [ \frac{{( - 1 )}^{α + 1} z}{{r}_{α} [ {r}_{α} + {d}_{α} ]} ] , \\ and$$

$${B}_{y 1} = \frac{{μ}_{0} {I}_{1}}{4 π} {\sum }_{α = 1}^{4} [ \frac{{( - 1 )}^{α + 1} z}{{r}_{α} [ {r}_{α} + {( - 1 )}^{α + 1} {C}_{α} ]} ] .$$

where a₁ and b₁ represent the sides of the rectangular loop of wire in the x-y plane. In the following equations, the side dimensions of the coils are denoted 2a₂ by 2b₂ as illustrated in FIG. 20A.

C1=[(a₂+(x−Sₓ)]

C2=[(a₂−(x−Sₓ)]

C3=−[(a₂−(x−Sₓ)]

C4=−[(a₂+(x−Sₓ)]

D1=y−S_{y}+b₂

D2=y−S_{y}+b₂

D3=y−S_{y}−b₂

D4=y−S_{y}−b1

R1=[(a₂+x−Sₓ)²+(y−S_{y}+b₂)²+z²]^{1/2}

R2=[(a₂−x+Sₓ)²+(y−S_{y}+b₂)²+z²]^{1/2}

R3=[(a₂−x+Sₓ)²+(y−S_{y}−b₂)²+z²]^{1/2}

R4=[(a₂+x−Sₓ)²+(y−S_{y}−b₂)²+z²]^{1/2}

Bx1=z/[R1·(R1+D1)]

Bx2=−z/[R2·(R2+D2)]

Bx3=z/[R3·(R3+D3)]

Bx4=−z/[R4·(R4+D4)]

Bx=Bx1+Bx2+Bx3+Bx4

By1=z/[R1·(R1+D1)]

By2=−z/[R2·(R2−D2)]

By3=z/[R3·(R3+D3)]

By4=−z/[R4·(R4−D4)]

By=By1+By2+By3+By4

Bz1=−D1/[R1·(R1+C1)]−C1/[R1·(R1+D1)]

Bz2=D2/[R2·(R2−C2)]−C2/[R2·(R2+D2)]

Bz3=−D3/[R3·(R3+C3)]−C3/[R3·(R3+D3)]

Bz4=D4/[R4·(R4−C4)]−C4/[R4·(R4+D4)]

Bz=Bz1+Bz2+Bz3+Bz4

Reference is made to FIG. 20B illustrating the detection of polar coordinates of an external emitter/marker (magnetic field source) by the corners of the coil loop. The marker could be inclined to the magnetic field vector; in terms of Cartesian coordinates the following relations hold:

x=r·Cos(θ)·Sin(φ), y=r·Sin(θ)·Sin(φ), z=r·Cos(φ)

where θ and φ represent the marker inclination angle to the x and z axes respectively.

The received marker signal is proportional to the scalar product of the marker magnetic vector of unity magnitude with the field magnetic vector:

B′(θ,φ)=|[Bx(θ,φ)+By(θ,φ)+Bz(φ)]|

and

B(θ,φ)=B′(θ,φ)*W(θ,φ)

where W(θ,φ)=[|[Σₖ Sign(B′ₖ(θ,φ)*B′ₖ²(θ,φ)]|}^{1/2}, and where the index k denotes the specific coils in the array antenna.

Therefore, for each of the antenna's loop, the received marker signal is the powering strength of the marker induced by all the antenna loops multiplied by the propagated wave amplitude generated from the local distortion of the magnetic field due to the marker loading effect.

The inventor has shown the compliance of the above theoretical model in several spatial positions and angular inclinations for antennas with square loops of 40 cm and 20 cm in size: for example, FIG. 20C illustrates the compliance of a measured marker signal 20M by an antenna coil as a function of the axial displacement along the main Z-axis, with a theoretical curve 20T based on the above theoretical model, for antennas with square loops of 40 cm in size. The compliance of the above theoretical model has also been shown for marker signals measured by one antenna coil as a function of the axial displacement along the X-axis in perpendicular to the main Z-axis, as a function of the angular inclination to the main Z-axis, as a function of the axial displacement along the X-axis in perpendicular to the main Z-axis and angularly inclined at Phi=+/−45° to the Z-axis. The compliance was also shown for marker signals measured by a plurality of sensing coils arranged in three arrays defining three sensing apertures located in three different non-parallel planes.

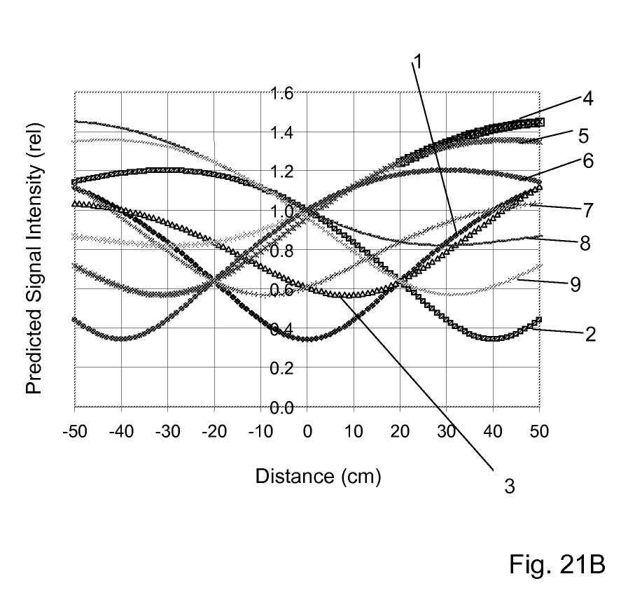

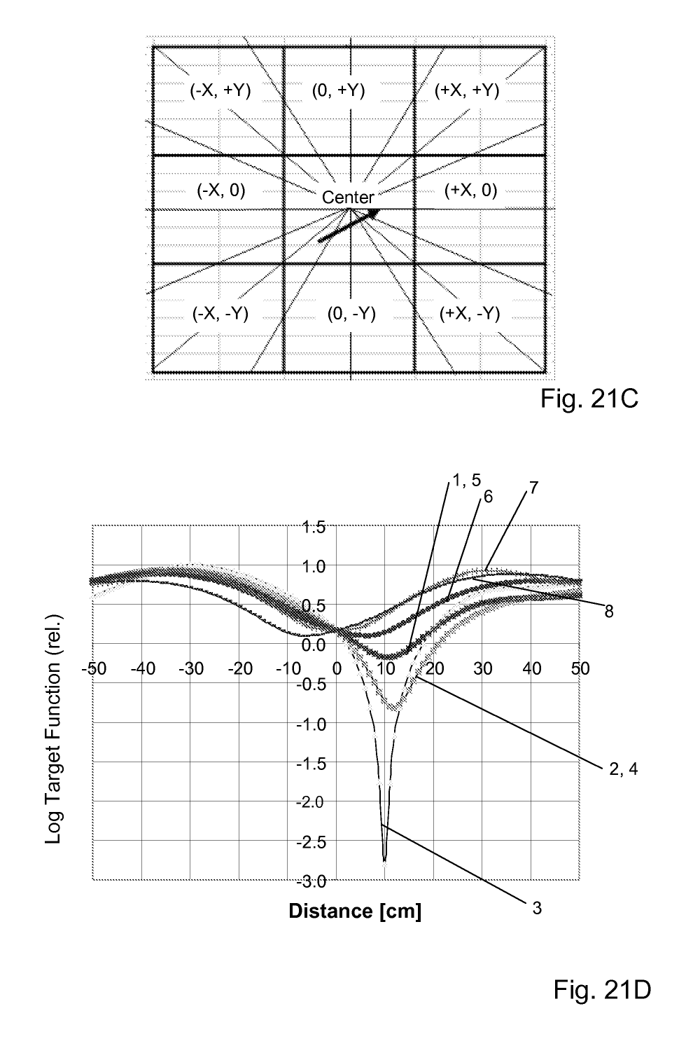

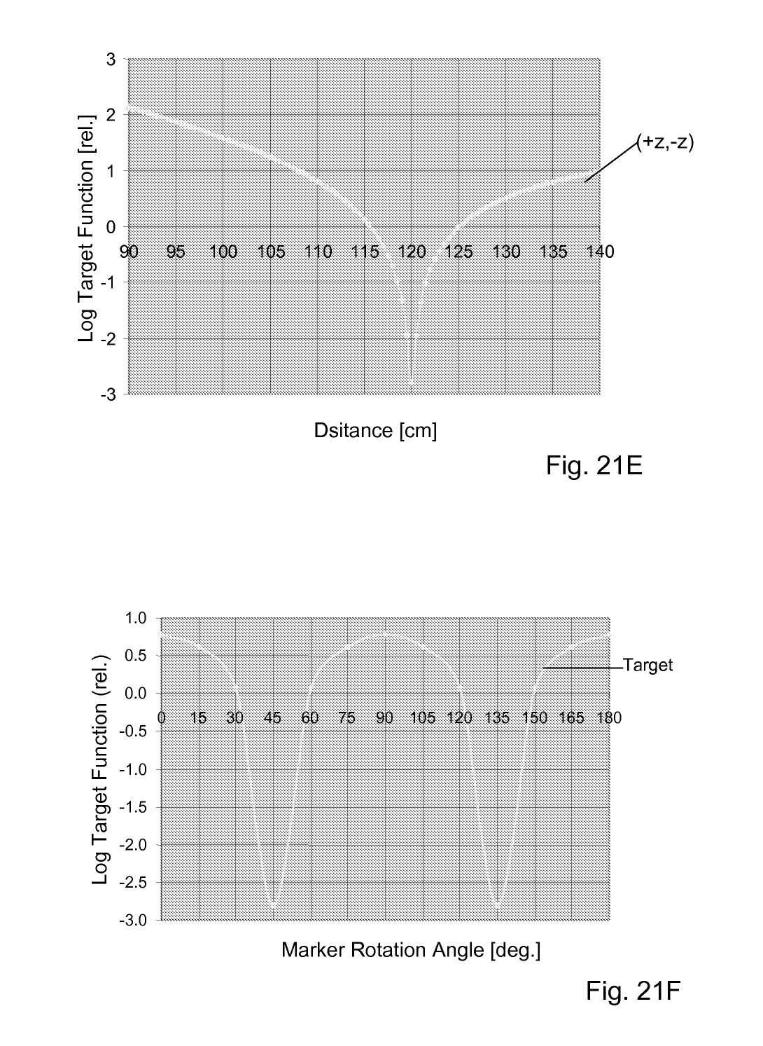

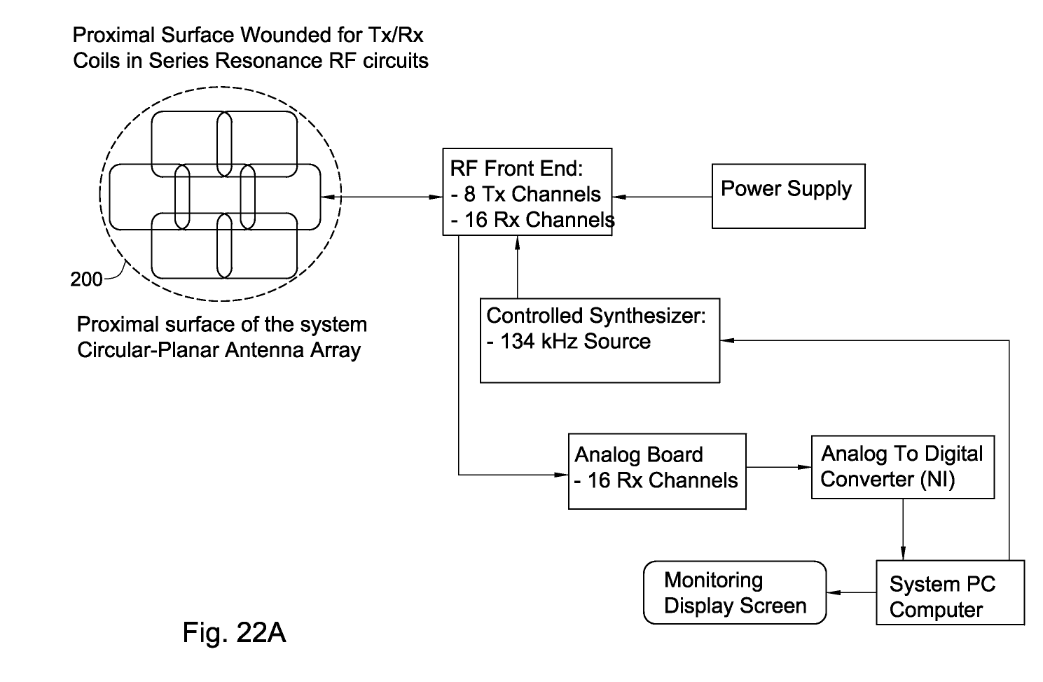

From the derived analytical expressions, the predicted signal intensities out of the phase array of square coils, in the presence of the marker in the antenna magnetic field at any field point (x,y,z) and any marker inclination (θ,φ) to the local magnetic field of each coil, is represented, for the case of nine coils in the planar array as shown in FIG. 21A, by the following predicted vector data (PVD):

PVD=[B_{θ,φ}(ctr), B_{θ,φ}(+x,0), B_{θ,φ}(+x,+y), B_{θ,φ}(0,+y), B_{θ,φ}(−x,0), B_{θ,φ}(−x,−y), B_{θ,φ}(0,−y), B_{θ,φ}(+x,−y)]

where the coil location (ctr) relates to the square coil that is centered at (0,0)

Reference is made to FIGS. 21A and 21B illustrating, respectively, a matrix of nine phase array coils and their coordinates, and predicted signal intensities to be detected by these phase array coils from a marker located at the field point (x,y,z)=(10,10,120) cm.

The predicted signal intensities from phase array coils including, for example nine coils, are designated by the following data vector:

[1 (0,0), 2 (+x,+y), 3 (0,+y), 4 (−x,+y), 5 (−x,0), 6 (−x,−y), 7 (0,−y), 8 (+x,−y), 9 (+x,0)]

The measured signal intensities from the phase array coils in the presence of the marker in the magnetic field region of the nine coils are represented by the following measured data vector (MDV):

MDV=[Bₘ(ctr), Bₘ(+x,+y), Bₘ(0,+y), Bₘ(−x,+y), Bₘ(−x,0), Bₘ(−x,−y), Bₘ(0,−y), Bₘ(+x,−y), Bₘ(+x,0)];

where the coil location (ctr) relates to the square coil that is centered at (0,0).

In order to identify the marker location, a Target Function (TF) is defined by the difference between the predicted and measured data vectors. The target function has the absolute minimum at the marker location and at a certain angle to the magnetic field vector produced by the phase array coils. Thus, the target function can be expressed as follows:

TF=[B_{θ,φ}(ctr)=Bₘ(ctr)]²+[B_{θ,φ}(+x,0)−Bₘ(+x,0)]²+[B_{θ,φ}(+x,+y)−Bₘ(+x,+y)]²+[B_{θ,φ}(0,+y)−Bₘ(0,+y)]²+[B_{θ,φ}(−x,+y)−Bₘ(−x,+y)]²+[B_{θ,φ}(−x,0)−Bₘ(−x,0)]²+[B_{θ,φ}(−x,−y)−Bₘ(−x,−y)]²+[B_{θ,φ}(0,−y)−Bₘ(0,−y)]²+[B_{θ,φ}(+x,−y)−Bₘ(+x,−y)]²

FIG. 21C shows the coils' matrix of FIG. 21A used for the Target Function considered in the calculations. The search for absolute minimum to the Target Function value is performed first along eight traces intercepting the perpendicular axis to the plane of the phase array coils, where these traces are marked as follows:

(+x,0), (+x,+y.5), (+x,+y), (+x.5,+y), (0,+y), (−x.5,+y), (−x,+y), (−x,+y.5).

Example of the obtained results for the Target Function value along the designated traces 1 (+x,0), 2 (+x,+y.5), 3 (+x,+y), 4 (+x.5,+y), 5 (0,+y), 6 (−x.5,+y), 7 (−x,+y), 8 (−x,+y.5), yields the graphs of FIGS. 21D-21F. These figures plot the target function vs the distance to the marker from the coils along 8 traces (FIG. 21D), the distance to the marker along the Z-axis for the (+x,+y) trace (FIG. 21E), and the marker rotation angle (FIG. 21F), all for the marker at (10, 10, 120) cm being 45° inclined to the magnetic field vector.

Alternatively, the minimum for the Target Function could also be searched by dividing the space around the implanted marker to cube of e.g. 3×3×3 sub-volumes or 5×5×5 sub-volumes. Then sub-dividing again in a similar manner the previous sub-volume of lowest Target Function, till convergence to the low Target Function value is achieved.

It should be understood that the above procedure for determining the Target Function is repeated for each of the other two planes, x-z and y-z planes, using the three arrays spaced from each other along the z-axis. The so-determined minimum Target Function values along the three axes are indicative of the marker's spatial location (coordinates along said axes), and the inclination angle to the magnetic field vector created by the phase array coils.

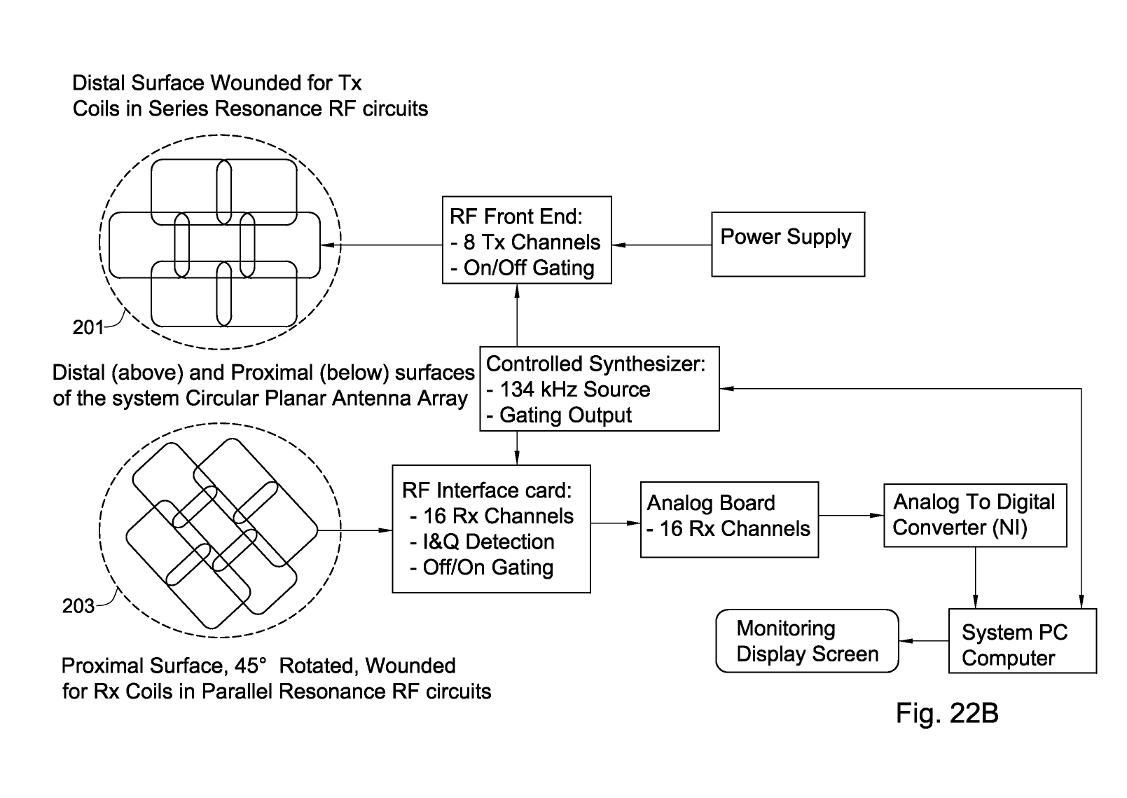

Reference is made to FIG. 22A illustrating the transmitting and receiving functional parts of the antenna system of the present invention. As described in details above, the antenna system of the present invention is a transceiver, for both transmitting an exciting electromagnetic field for activating a location marker and receiving a signal therefrom. The operation of these two essentials functions of the antenna system of the present invention are illustrated in the figure. In this specific configuration, the antenna system comprises a transmitting/receiving antenna array 200 configured as a circular planar antenna array having a diameter of 120 cm and comprising 7 square closed-loop coils of 40×40 cm where each antenna coil is electronically designed in a series resonance circuit. The antenna array also comprises a power supply energizing a RF front end for activating the 8 channels (7 of each coil plus one spare) of the transmitting array and the 16 channels of the receiving array (2 of each coil plus two spare), an analog board unit for amplification and filtering of the 16 receiving channels, a synthesizer of generating an RF high frequency signal of 134 KHz in this specific example and a processor unit. The processor unit includes an analog-to-digital converter, and a data processing and analyzing utility. The processor unit may include other utilities, such as memory, display, etc.

Reference is made to FIG. 22B, illustrating another example of the configuration of the antenna system of the present invention, in which the excitation and receiving sessions are timely separated, namely exciting high frequency signal and the received low frequency signal are not transmitted/received simultaneously but at a certain alternating rate, to reduce the transmitting noise signal generated during the receiving signal acquisition. In this specific configuration, the antenna system is configured as a circular planar antenna array having two surfaces, the distal surface (with respect to a zone from which a marker is to be detected) comprising a transmitting antenna array (Tx) 201 and the proximal surface comprising a receiving antenna array (Rx) 203. wherein the transmitting antenna array 201 and the receiving antenna array 203 operates successively at a certain rate. The transmitting antenna array 201 is configured as a circular planar antenna array having a diameter of 120 cm and comprises 7 square closed-loop coils of 40×40 cm where each antenna coil is electronically designed in a series resonance circuit. The receiving antenna array 203 is also configured as a circular planar antenna array having a diameter of 120 cm and comprises 7 square closed-loop coils of 40×40 cm where each coil is electronically designed in a parallel resonance circuit. In this case, the RF Front End operates the transmitting channels at a certain rate synchronized with the receiving channels. For facilitating the transmitting/receiving operations, the receiving antenna array may be rotated at a certain angle, e.g. 45°, respectively to the transmitting antenna array.

Those skilled in the art will readily appreciate that various modifications and changed can be applied to the embodiments of the invention as hereinbefore described without departing from its scope defined in and by the appended claims.

1. A location marker comprising an electronic circuit comprising a magnetic component and an electronic switch unit electrically connected to said magnetic component, said electronic circuit being switchable from its normally inoperative state into its operative state by a high radio frequency electromagnetic field, said electronic circuit while in the operative state thereof generating a predefined periodic distortion pattern of a certain fixed frequency relatively low as compared to said high radio frequency thus inducing a corresponding modulation of said electromagnetic field.

2. A location marker according to claim 1, wherein the high frequency/low frequency ratio is at least 2.

3. A location marker according to claim 2, wherein the high frequency is between about 100 to 150 KHz.

4. A location marker according to claim 2, wherein the low frequency is in the range of about 1 to 20 KHz.

5. A location marker according to claim 1, wherein the magnetic component is energizable by the high frequency field to actuate the electronic switch unit which is programmed to operate in a switching mode of said predefined periodic low frequency modulation, the switching operation causes said low frequency distortion pattern of the electric current flow through the magnetic component.

6. A location marker according to claim 1, wherein said magnetic component comprises a ferromagnetic element and a coil with passive and active magnetic properties and an electronic switch unit affecting the active magnetic properties in said periodic low frequency distortion pattern.

7. A location marker according to claim 1, wherein said electronic circuit comprises a loading RF resonance circuit with passive and active magnetic properties and an electronic switch unit affecting the active magnetic properties in said periodic low frequency distortion pattern.

8. A system for locating a target, the system comprising:
one or more location markers for placing at the target, each of the location markers being that as defined in claim 1 and generating a predefined periodic pattern of a different low frequency; and
a transceiver antenna system comprising a plurality of sensing coils arranged in three arrays defining three sensing apertures located in three different non-parallel planes for (i) generating and receiving said high frequency electromagnetic field, (ii) processing the received signal and extracting the low frequency periodic component therefrom, one for each marker, and (iii) identifying location of the marker based on the extracted component.

9. A system according to claim 8, wherein said extracting comprises applying an autocorrelation function to the received signal.

10. A system according to claim 8, wherein each frequency pattern differs from the other by at least one of the frequency and amplitude of said distortion pattern.

11. A system according to claim 8, wherein the sensing aperture of the sensing coil is partially overlapping with that of an adjacent coil, setting their mutual inductance to zero.

12. A system according to claim 8 , comprising a magnetic field source configured and operable for generating an AC magnetic field of a frequency of about 100 KHz-150 KHz.

13. A system according to claim 8, wherein the high radio frequency electromagnetic field has a constant frequency and peak amplitude.

14. A system according to claim 8, wherein said marker is implantable.

15. A system according to claim 8, for directing irradiation to a target tissue.

16. A system according to claim 8, wherein said marker is fitted on a medical tool.

17. A system according to claim 16, wherein said medical tool is a catheter, endoscope or needle.

18. A medical instrument carrying a location marker according to claim 1.

19. A transceiver antenna system comprises a magnetic field source of an alternating electromagnetic field; a plurality of sensing coils arranged in three arrays defining three sensing apertures located in three different non-parallel planes, wherein sensing apertures of locally adjacent coils in each array are partially overlapping setting their mutual inductance to zero; and, a receiver for receiving an incoming electromagnetic signal and extracting therefrom a predefined periodic signal of a low frequency, thereby enabling identification and determination of a three-dimensional location of the marker with a high signal-to-noise ratio.

20. An antenna system comprising: a receiving antenna arrangement comprising three or more phase arrays of closed-loop coils defining each a receiving aperture of substantially the same polygonal geometry; said arrays being each located in a different plane, all of the planes being in a spaced-apart parallel relationship, said coils being arranged with reduced cross-talk between at least some of said coils.

21. An antenna system according to claim 20, consisting of three of said phase arrays.

22. An antenna system according to claim 20, wherein said coils are arranged such that each coil of one array is superimposed along at least two sides thereof by coils from at least one other array with certain polygonal overlapping regions substantially small as compared to said receiving aperture.

23. An antenna system according to claim 20, wherein each coil of a first of the arrays is along two sides thereof superimposed by two coils, one from each of a second and a third of the arrays.

24. An antenna system according to claim 20 comprising a receiver unit, configured and operable for processing received signals from each of said coils and determining three-dimensional coordinates of an external source of an electromagnetic field.

25. An antenna system according to claim 24, wherein said receiver unit is configured and operable for processing received signals from each of said coils and determining three-dimensional coordinates of an external source of an electromagnetic field by searching for a minimum to a target function value performed along traces intercepting the perpendicular axis to the phase array coils.

26. An antenna system according to claim 24, wherein said receiver unit is configured and operable for processing received signals from each of said coils and determining three-dimensional coordinates of an external source of an electromagnetic field by determining a predicted vector data indicative of the intensities of said external source signal detected by the phase array coils in the antenna magnetic field at any field point and at any inclination of the external source relatively to each local magnetic field of each coil;
detecting the signal intensities emitted by said external source by said phase coil arrays and generating a measured data vector; and
identifying the location of said external magnetic source by defining a target function indicative of the difference between said predicted vector data and said measured data vector, the target function having a minimum at the external source location and at a certain angle to the magnetic field vector produced by the phase array coils.

27. An antenna system according to claim 25, wherein the minimum of the target function is obtained by dividing the space around said external source to a cube of sub-volumes and by sub-dividing again in a similar manner the previous sub-volume of lowest target function, until a convergence to a low target function value is achieved.

28. An antenna system according to claim 20, comprising a transmitting antenna arrangement.

29. An antenna system according to claim 28, wherein said transmitting antenna arrangement comprises said phase arrays of coils each generating an electromagnetic field.

30. An antenna system according to claim 24, wherein the receiver unit configured and operable for processing received signals from each of said coils comprises an isolated splitter electrical circuit for filtering out an electromagnetic component of the transmitted field from the received signals that are to be processed.

31. An antenna system according to claim 24, wherein the receiver unit configured and operable for processing received signals from each of said coils enables at least one of the following: elimination of a pre-calibration stage: a phase-shifting adjustment and a phase cancellation.

32. An antenna arrangement for use in a system according to claim 20.
